SEQUENCE LISTING (49 sequences)

SEQ ID NO: 1 - protein, 1194 aa, Homo sapiens
METRRRLEQERATMQMTPGEFRRPRLASFGGMGTTSSLPSFVGSGNHNPAKHQLQNGYQGNGDYGSYAPAAPTTSSMGSSIRHSPLSSGISTPVTNVSPMHLQHIREQMAIALKRLKELEEQVRTIPVLQVKISVLQEEKRQLVSQLKNQRAASQINVCGVRKRSYSAGNASQLEQLSRARRSGGELYIDYEEEEMETVEQSTQRIKEFRQLTADMQALEQKIQDSSCEASSELRENGECRSVAVGAEENMNDIVVYHRGSRSCKDAAVGTLVEMRNCGVSVTEAMLGVMTEADKEIELQQQTIEALKEKIYRLEVQLRETTHDREMTKLKQELQAAGSRKKVDKATMAQPLVFSKVVEAVVQTRDQMVGSHMDLVDTCVGTSVETNSVGISCQPECKNKVVGPELPMNWWIVKERVEMHDRCAGRSVEMCDKSVSVEVSVCETGSNTEESVNDLTLLKTNLNLKEVRSIGCGDCSVDVTVCSPKECASRGVNTEAVSQVEAAVMAVPRTADQDTSTDLEQVHQFTNTETATLIESCTNTCLSTLDKQTSTQTVETRTVAVGEGRVKDINSSTKTRSIGVGTLLSGHSGFDRPSAVKTKESGVGQININDNYLVGLKMRTIACGPPQLTVGLTASRRSVGVGDDPVGESLENPQPQAPLGMMTGLDHYIERIQKLLAEQQTLLAENYSELAEAFGEPHSQMGSLNSQLISTLSSINSVMKSASTEELRNPDFQKTSLGKITGNYLGYTCKCGGLQSGSPLSSQTSQPEQEVGTSEGKPISSLDAFPTQEGTLSPVNLTDDQIAAGLYACTNNESTLKSIMKKKDGNKDSNGAKKNLQFVGINGGYETTSSDDSSSDESSSSESDDECDVIEYPLEEEEEEEDEDTRGMAEGHHAVNIEGLKSARVEDEMQVQECEPEKVEIRERYELSEKMLSACNLLKNTINDPKALTSKDMRFCLNTLQHEWFRVSSQKSAIPAMVGDYIAAFEAISPDVLRYVINLADGNGNTALHYSVSHSNFEIVKLLLDADVCNVDHQNKAGYTPIMLAALAAVEAEKDMRIVEELFGCGDVNAKASQAGQTALMLAVSHGRIDMVKGLLACGADVNIQDDEGSTALMCASEHGHVEIVKLLLAQPGCNGHLEDNDGSTALSIALEAGHKDIAVLLYAHVNFAKAQSPGTPRLGRKTSPGPTHRGSFD

SEQ ID NO: 2 - DNA, 20 bp, Artificial Sequence
  Description of Artificial SequenceSynthetic DNA
tactttgtggagacccccta

SEQ ID NO: 3 - DNA, 21 bp, Artificial Sequence
  Description of Artificial SequenceSynthetic DNA
gcttgtcgtgcccatgcctcc

SEQ ID NO: 4 - DNA, 20 bp, Artificial Sequence
  Description of Artificial SequenceSynthetic DNA
cactggggtggagatccctg

SEQ ID NO: 5 - DNA, 20 bp, Artificial Sequence
  Description of Artificial SequenceSynthetic DNA
attatggtagctatgcccca

SEQ ID NO: 6 - DNA, 22 bp, Artificial Sequence
  Description of Artificial SequenceSynthetic DNA
tgcagcacatccgcgagcagat

SEQ ID NO: 7 - DNA, 18 bp, Artificial Sequence
  Description of Artificial SequenceSynthetic DNA
tccggcaacttacagcag

SEQ ID NO: 8 - DNA, 20 bp, Artificial Sequence
  Description of Artificial SequenceSynthetic DNA
cagctgtgaggcctcctcag

SEQ ID NO: 9 - DNA, 22 bp, Artificial Sequence
  Description of Artificial SequenceSynthetic DNA
gcctctgtggtacacgacgatg

SEQ ID NO: 10 - DNA, 23 bp, Artificial Sequence
  Description of Artificial SequenceSynthetic DNA
aggcatctcctgccagcctgaat

SEQ ID NO: 11 - DNA, 22 bp, Artificial Sequence
  Description of Artificial SequenceSynthetic DNA
tccacagacctcccagcacatc

SEQ ID NO: 12 - DNA, 29 bp, Artificial Sequence
  Description of Artificial SequenceSynthetic DNA
tctgtgttgctgcctgtttcgcagacgct

SEQ ID NO: 13 - DNA, 23 bp, Artificial Sequence
  Description of Artificial SequenceSynthetic DNA
agacaagtgttggtgcaggactc

SEQ ID NO: 14 - DNA, 17 bp, Artificial Sequence
  Description of Artificial SequenceSynthetic DNA
ggacagtagctgtagga

SEQ ID NO: 15 - DNA, 22 bp, Artificial Sequence
  Description of Artificial SequenceSynthetic DNA
cagctgatggcctgtcaaaccc

SEQ ID NO: 16 - DNA, 22 bp, Artificial Sequence
  Description of Artificial SequenceSynthetic DNA
gggttcctcagctcttcagtgc

SEQ ID NO: 17 - DNA, 22 bp, Artificial Sequence
  Description of Artificial SequenceSynthetic DNA
tcctcattcccaggtcctcagg

SEQ ID NO: 18 - DNA, 23 bp, Artificial Sequence
  Description of Artificial SequenceSynthetic DNA
cagtcctagcatcacacactctg

SEQ ID NO: 19 - DNA, 18 bp, Artificial Sequence
  Description of Artificial SequenceSynthetic DNA
tcctgccaatgactgtga

SEQ ID NO: 20 - DNA, 26 bp, Artificial Sequence
  Description of Artificial SequenceSynthetic DNA
gggtgtgagttttcatttttattgcc

SEQ ID NO: 21 - DNA, 24 bp, Artificial Sequence
  Description of Artificial SequenceSynthetic DNA
actgacagcattagcctctagaac

SEQ ID NO: 22 - DNA, 23 bp, Artificial Sequence
  Description of Artificial SequenceSynthetic DNA
tgagcacaccttgcatctcctga

SEQ ID NO: 23 - DNA, 21 bp, Artificial Sequence
  Description of Artificial SequenceSynthetic DNA
cattaaatgtgggaggggcaa

SEQ ID NO: 24 - DNA, 24 bp, Artificial Sequence
  Description of Artificial SequenceSynthetic DNA
tcttcttgtgaccaatcgtaactt

SEQ ID NO: 25 - DNA, 23 bp, Artificial Sequence
  Description of Artificial SequenceSynthetic DNA
tacacactggggatggtgtttgc

SEQ ID NO: 26 - DNA, 25 bp, Artificial Sequence
  Description of Artificial SequenceSynthetic DNA
aatagaagaactaacgaccacttgg

SEQ ID NO: 27 - DNA, 23 bp, Artificial Sequence
  Description of Artificial SequenceSynthetic DNA
ttagagaagagagggtggaaggg

SEQ ID NO: 28 - DNA, 23 bp, Artificial Sequence
  Description of Artificial SequenceSynthetic DNA
agaaggggctgcttcctaagaga

SEQ ID NO: 29 - DNA, 22 bp, Artificial Sequence
  Description of Artificial SequenceSynthetic DNA
gggtgcattcctgagcacagga

SEQ ID NO: 30 - DNA, 24 bp, Artificial Sequence
  Description of Artificial SequenceSynthetic DNA
cagtacgtacttctgaagtccttg

SEQ ID NO: 31 - DNA, 21 bp, Artificial Sequence
  Description of Artificial SequenceSynthetic DNA
tcccagagctcccgtccagag

SEQ ID NO: 32 - DNA, 24 bp, Artificial Sequence
  Description of Artificial SequenceSynthetic DNA
gagaaacccaacatggcttgttct

SEQ ID NO: 33 - DNA, 21 bp, Artificial Sequence
  Description of Artificial SequenceSynthetic DNA
ggggtccaccagtctggtgga

SEQ ID NO: 34 - DNA, 24 bp, Artificial Sequence
  Description of Artificial SequenceSynthetic DNA
tgaggtcacttattaacccccagt

SEQ ID NO: 35 - DNA, 23 bp, Artificial Sequence
  Description of Artificial SequenceSynthetic DNA
gtatctgtcaccccaacaggaac

SEQ ID NO: 36 - DNA, 20 bp, Artificial Sequence
  Description of Artificial SequenceSynthetic DNA
cagatgtggtcctgggttct

SEQ ID NO: 37 - DNA, 25 bp, Artificial Sequence
  Description of Artificial SequenceSynthetic DNA
tcagtcaaggtcacagtcatattaa

SEQ ID NO: 38 - DNA, 22 bp, Artificial Sequence
  Description of Artificial SequenceSynthetic DNA
ttgtgctgtctgtcagcatatg

SEQ ID NO: 39 - DNA, 24 bp, Artificial Sequence
  Description of Artificial SequenceSynthetic DNA
aagtaaatgtgacaggtaaaaagg

SEQ ID NO: 40 - DNA, 25 bp, Artificial Sequence
  Description of Artificial SequenceSynthetic DNA
cttgacacagtattttcagcttttg

SEQ ID NO: 41 - DNA, 20 bp, Artificial Sequence
  Description of Artificial SequenceSynthetic DNA
gaattccttcctcccctgtc

SEQ ID NO: 42 - DNA, 20 bp, Artificial Sequence
  Description of Artificial SequenceSynthetic DNA
aaaccaggcacaatcaaacc

SEQ ID NO: 43 - DNA, 29 bp, Artificial Sequence
  Description of Artificial SequenceSynthetic DNA
gtggagaccaggacaaggaacagaaagac

SEQ ID NO: 44 - DNA, 21 bp, Artificial Sequence
  Description of Artificial SequenceSynthetic DNA
tccagagggggaggtggcttt

SEQ ID NO: 45 - DNA, 21 bp, Artificial Sequence
  Description of Artificial SequenceSynthetic DNA
gcagctgtgaggcctcctcag

SEQ ID NO: 46 - DNA, 22 bp, Artificial Sequence
  Description of Artificial SequenceSynthetic DNA
tccacagacctcccagcacatc

SEQ ID NO: 47 - DNA, 21 bp, Artificial Sequence
  Description of Artificial SequenceSynthetic DNA
aagaagagaaaaggtagttgg

SEQ ID NO: 48 - DNA, 21 bp, Artificial Sequence
  Description of Artificial SequenceSynthetic DNA
ctattaaaactcaatttcttt

SEQ ID NO: 49 - DNA, 24 bp, Artificial Sequence
  Description of Artificial SequenceSynthetic DNA
cctaaaacctctataatacacaac

(19) United States
(12) Patent Application Publication
(10) Pub. No.: US 2007/0128645 A1
(43) Pub. Date: Jun. 7, 2007

(54) Use of KIAA0172 gene in treatment and diagnosis of diseases as well as in pharmaceutical development

(75) Inventors: Ryoichi Kiyama, Tsukuba-shi (JP); Keisuke Kitajima, Tsukuba-shi (JP); Shinobu Oguchi, Tsukuba-shi (JP); Michio Oishi, Kisarazu-shi (JP); Osamu Ohara, Kisarazu-shi (JP); Takahiro Nagase, Kisarazu-shi (JP)

Correspondence Address: FOLEY AND LARDNER LLP;SUITE 500, 3000 K STREET NW, WASHINGTON, DC, 20007, US

(73) Assignees: National Institute of Advanced Industrial Science; Info Genes Co., Ltd.; Kazusa DNA Research Institute

(21) Appl. No.: 11/606,148

(22) Filed: Nov. 30, 2006

Related U.S. Application Data
(62) Division of application No. 10/509,738, filed on May 18, 2005, now abandoned.

(30) Foreign Application Priority Data
Apr. 1, 2002 (JP) 2002-99422

(51) Int. Cl. C12Q 1/68; C12P 19/34

(52) U.S. Cl. 435/6; 435/91.2

(57) ABSTRACT

An object of the present invention is to provide use of the KIAA0172 gene in treatment and diagnosis of diseases as well as in pharmaceutical development. An agent for treating cancer which comprises as an active ingredient a polypeptide encoded by the KIAA0172 gene, a partial sequence thereof or a variant thereof; an agent for treating cancer which comprises as an active ingredient an oligonucleotide including the KIAA0172 gene sequence; an agent for detecting cancer which comprises an antibody which recognizes polypeptide encoded by the KIAA0172 gene; an agent for detecting cancer which comprises an oligonucleotide including the KIAA0172 gene sequence; a composition for treating cancer which comprises said agent for treating cancer and a pharmaceutically acceptable carrier; and a composition for detecting cancer which comprises said agent for detecting cancer and a pharmaceutically acceptable carrier.

This application is a divisional application of U.S. application Ser. No. 10/509,738 filed May 18, 2005; which is the U.S. National Phase of PCT/JP02/07622 filed Jul. 26, 2002; which claims priority from Japanese Application No. 2002-99422 filed Apr. 1, 2002, the contents of which are relied upon and incorporated by reference.

### TECHNICAL FIELD

The present invention relates to use of KIAA0172 gene having function of suppressing proliferation of cancer cells in treatment and diagnosis of diseases as well as in pharmaceutical development.

### BACKGROUND ART

KIAA0172 gene encodes a protein having a molecular weight of about 140 kDa. The cDNA had already been cloned and the cDNA nucleotide sequence was reported almost in full length (Nagase et al., DNA Res.3 (1), 17-24 (1996)). The KIAA0172 gene was located on BAC clone RPCI-11-130C19 mapped to chromosome 9p24. The cDNA nucleotide sequence reported is found in GenBank database under accession number D79994 (registered as: Human mRNA for KIAA0172 gene, partial cds.). However, the function of the KIAA0172 gene remains unknown.

### DISCLOSURE OF THE INVENTION

An object of the present invention is to provide use of the KIAA0172 gene in treatment and diagnosis of diseases as well as in pharmaceutical development.

The present inventors had performed structural analysis of the KIAA0172 gene and found that the gene is located on BAC clone RPCI-11-130C19 mapped to chromosome 9p24 existing between the nucleotide numbers 85563-120956 on this clone. We had further conducted comparison of this BAC clone with the cDNA reported by Nagase et al. (Nagase et al., DNA Res.3, 17-24, 1996) and determination of cap sites (see Example 1) and thereby found that it has a gene structure comprising ten (10) exons. Then we had conducted functional analysis of the KIAA0 172 gene whose function has been unknown and consequently found that the KIAA0 172 gene possesses function of suppressing proliferation of cancer cells and transforming activity. We also found that the expression of the gene was often suppressed in renal cancer patients and demonstrated that this gene has regulating function in cell proliferation and, when it was lost, certain cancer-specific characters appeared.

First, we have found that LOH (Loss of Heterozygosity) occurs at high frequency in the genome region containing the KIAA0172 gene in LOH analysis. Then we had conducted RT-PCR analysis to analyze the expression of the KIAA0172 gene in normal and renal cancer tissues and found that the transcription of the KIAA0 172 gene is significantly decreased in the renal cancer tissues. Furthermore, we determined nucleotide sequences of exons 1-10 derived from cancer tissues, compared them with the KIAA0172 cDNA structure (accession number D79994) registered in GenBank, and found that there exist mutations in nucleotide sequences associated with changes in the amino acid sequence of the KIAA0172 gene products in the renal cancer tissues. The rate of the nucleotide mutation was significantly above the frequency of the spontaneous mutation of a gene, suggesting the association between the genetic mutation of KIAA0172 gene and the formation of renal cancer. In addition, we also investigated occurrence and sites of methylation, which had already been well known as a common mechanism of suppressing gene expression, and experimentally demonstrated that allele-specific methylation occurrs both in the renal cancer and normal tissues and that the methylation suppressed KIAA0172 gene expression. Taken together,=the methylation which takes place in KIAA0172 gene is a risk-factor. In addition, it has been revealed that single nucleotide polymorphism can be used for prediction (diagnosis) of KIAA0172 gene expression in each patient.

On the other hand, homology search of the KIAA0172 gene with known nucleotide sequences revealed that KIAA0172 and ankyrin, or DAPK protein, share part of their gene structures. According to the results of two-hybrid experiments, the domain homologous to ankyrin is a site interacting with other gene products.

In the meantime, some characteristics of KIAA0172 gene products, such as cytoplasmic and tissue localization and molecular weight heterogeneity, have been determined using immunostaining, immunoprecipitation, and western analysis using a KIAA0 1 72-specific polyclonal antibody.

Inventors considered that use of this gene enables diagnosis (evaluation of risk-factors and stage of cancer progression) and gene therapy of the cancer, which takes advantage of the KIAA0172 function of suppressing cancer growth, and, therefore, the present invention which utilizes the KIAA0172 gene is to treat or diagnose renal cancer. In addition, we supposed that this gene, expression of which has been observed in a number of other tissues, also has a role in cell proliferation in non-renal tissues, thereby it can be used for therapeutic treatment and diagnosis of cancer even in tissues other than kidney.

That is, the present invention provides the following:

(1) An agent for treating cancer which comprises as an active ingredient a polypeptide encoded by KIAA0172 gene, a partial sequence thereof or a variant thereof;

(2) An agent for treating cancer which comprises as an active ingredient an oligonucleotide including KIAA0172 gene sequence, a part thereof or a variant thereof;

(3) An agent for detecting cancer which comprises an antibody which recognizes a polypeptide encoded by KIAA0172 gene;

(4) An agent for detecting cancer which comprises an oligonucleotide including KIAA0172 gene sequence, a part thereof or a variant thereof;

(5) A composition for treating cancer which comprises the agent for treating cancer according to (1) or (2) and a pharmaceutically acceptable carrier;

(6) A composition for detecting cancer which comprises the agent for detecting cancer according to (3) or (4) and a pharmaceutically acceptable carrier;

(7) A vector for treating cancer which comprises KIAA0172 gene, a partial sequence thereof or a variant thereof;

(8) A vector for detecting cancer which comprises KIAA0172 gene, a partial sequence thereof or a variant thereof;

(9) A method for detecting cancer using an antibody which recognizes KIAA0172 protein;

(10) The method for detecting cancer according to (9), which comprises a step of contacting a sample with an antibody which recognizes a polypeptide encoded by KIAA0172 gene;

(11) The method for detecting cancer according to (9) or (10), wherein said method is immunostaining using a tissue section;

(12) The method for detecting cancer, which comprises a step of contacting a sample with an oligonucleotide containing KIAA0172 gene sequence;

(13) A modified KIAA0172 gene or a fragment thereof having at least one of the following mutations (a) to (h):

(a) Mutation from CAC to CAG at the 52nd codon

(b) Mutation from GCG to GTG at the 168th codon

(c) Insertion of 6 nucleotides GCTGTA between the 268th and the 269th codons

(d) Mutation from GTA to GGA at the 269th codon

(e) Mutation from GAG to CAG at the 274th codon

(f) Mutation from TCC to GCC at the 306th codon

(g) Mutation from GCA to GTA at the 506th codon

(h) Mutation from CGT to CAT at the 509th codon;

(14) An agent for detecting cancer which comprises the modified KIAA0172 gene according to (13);

(15) The method for detecting cancer, which comprises a step of contacting a sample with an oligonucleotide containing the modified KIAA0172 gene sequence according to (13) or a part thereof;

(16) A detecting method for evaluating the risk of suffering from cancer, which comprises a step of contacting a sample with an oligonucleotide containing the modified KIAA0 172 gene sequence according to (13) or a part thereof;

(17) A gene fragment containing at least one of the following single nucleotide polymorphism sites (i) to (r) on the KIAA0172 gene:

(i) T/G polymorphism site for the third nucleotide of the codon number 273

(j) G/C polymorphism site for the third nucleotide of the codon number 299

(k) C/T polymorphism site for the first nucleotide of the codon number 372

(l) T/G polymorphism site for the third nucleotide of the codon number 380

(m) T/G polymorphism site for the third nucleotide of the codon number 497

(n) C/T polymorphism site for the third nucleotide of the codon number 453

(o) C/T polymorphism site for the third nucleotide of the codon number 478

(p) G/T polymorphism site for the third nucleotide of the codon number 507

(q) C/T polymorphism site for the third nucleotide of the codon number 1003 and

(r) G/C polymorphism site for the third nucleotide of the codon number 1120.

(18) A method for evaluating the risk of suffering from cancer, which comprises determining the respective nucleotides at the following single nucleotide polymorphism sites

(i) to (r) on the KIAA0172 gene:

(i) T/G polymorphism site for the third nucleotide of the codon number 273

(j) G/C polymorphism site for the third nucleotide of the codon number 299

(k) C/T polymorphism site for the first nucleotide of the codon number 372

(l) T/G polymorphism site for the third nucleotide of the codon number 380

(m) T/G polymorphism site for the third nucleotide of the codon number 497

(n) C/T polymorphism site for the third nucleotide of the codon number 453

(o) C/T polymorphism site for the third nucleotide of the codon number 478

(p) G/T polymorphism site for the third nucleotide of the codon number 507

(q) C/T polymorphism site for the third nucleotide of the codon number 1003 and

(r) G/C polymorphism site for the third nucleotide of the codon number 1120.

(19) A method for evaluating the risk of suffering from cancer, which comprises analyzing LOH (Loss of Heterozygosity) in the genome region including KIAA0172 gene;

(20) A method for evaluating the risk of suffering from cancer according to (19), which comprises determining loss of heterozygosity in either one or both of D9S 1779 and D9S 1858 which are microsatellite markers of chromosome 9p24 site;

(21) A method for evaluating the risk of suffering from cancer, which comprises analyzing methylation of KIAA0172 gene,

(22) The method for evaluating the risk of suffering from cancer according to (21), which comprises determining a methylation pattern of one or more CpG sequences present in KIAA0172 gene; and

(23) The method for evaluating the risk of suffering from cancer according to (22), in which the CpG sequence(s) is a CpG sequence(s) in the CpG island present in the 1st exon of KIAA0 172 gene.

Hereafter, the present invention is explained in detail.

Preparation of cDNA library, cloning and screening of a gene, and determination of a nucleotide sequence, etc. can be performed according to the state-of-the-art procedures such as J. Sambrook, E. F. Fritsch & T. Maniatis: Molecular Cloning, a laboratory manual, second edition, Cold Spring Harbor Laboratory Press (1989) and Ed Harlow and David Lanc: Antibodies, a laboratory manual, Cold Spring Harbor Laboratory Press (1988).

The gene of the present invention can be isolated by extracting MRNA and synthesizing cDNA. Human cells, such as human undifferentiated myeloid cell line KG-1 can be used as a source of mRNA. Preparation of MRNA can be performed by extracting total RNA by guanidine thiocyanate/cesium chloride method followed by affinity column method using oligo(dT)-cellulose, poly(U)-sepharose or the like, or alternatively a one-step batch process for obtaining poly(A)+RNA (mRNA). The thus obtained mRNA is used as a template to synthesize a single-stranded cDNA using an oligo(dT) primer and reverse transcriptase and a double-stranded cDNA is synthesized from the single-stranded cDNA.

The synthesized double-stranded cDNA can be incorporated into a suitable vector, which is used for the transformation of E. coli etc. to prepare a cDNA library which in turn can be used to obtain a part of the gene of the present invention. Then plaque hybridization using a probe synthesized based on a partially known sequence of the gene (EST reported as WI-12779), colony hybridization, immunoscreening, etc. can be used to obtain a targeted cDNA. The obtained cDNA fragment can be amplified by PCR method, and the nucleotide sequence thereof can be determined by Maxam-Gilbert method (Maxam, A. M. and Gilbert, W., Proc. Natl. Acad. Sci. USA., 74, 560, 1977), dideoxy method (Messing, J. et al., Nucl. Acids Res., 9, 309, 1981), or the like. Alternatively, the nucleotide sequence can be obtained from database as the cDNA nucleotide sequence of KIAA0172 gene had already been reported in almost full length (Nagase et al., DNA Res. 3, 17-24, 1996, registered under accession number D79994 and the designation as: Human mRNA for KIAA0172 gene, partial cds., in GenBank)).

The KIAA0172 gene consists of 10 exons, and the analysis of exons can be performed either by exon trapping method or based on the known partial gene information about the KIAA0172 gene.

The 5′-end information needed to determine a full length cDNA sequence can be obtained by performing a primer extension reaction.

The full length sequence of the KIAA0172 gene can be obtained based on the disclosure of the present specification and the above-mentioned known information refer to the KIAA0172 gene.

The KIAA0172 gene used in the present invention may be either a full length sequence or a partial sequence. The KIAA0172 gene utilized in the present invention also includes the full length DNA sequence containing introns as well as the DNA sequence only containing exon sites. An example of a partial sequence is one considered to be the functional site for the present gene such as ankyrin domain. The present gene also includes any variant DNA which can hybridize with the full length sequence of the KIAA0172 gene or a partial sequence thereof under a stringent condition. The stringent condition refers to a condition in which so-called specific hybrids are formed and non-specific hybrids are not. Examples of such a condition include those in which DNAs with high homology, i.e., DNAs which have homology of 60% or more, preferably 80% or more, hybridize with each other while nucleic acids with lower homology do not. More specifically, it means a condition having a sodium concentration of 150 to 900 mM, preferably 600 to 900 mM and a temperature of 60 to 68° C., preferably of 65° C.

In order to introduce a mutation into a gene, any known method such as Kunkel method, Gapped duplex method, and a method like these can be adopted. For example, a mutation may be introduced using a mutation introducing kit utilizing site-directed mutagenesis (for example, Mutant-K and Mutant-G, both are product of TAKARA Co., Ltd.) or LA PCR in vitro Mutagenesis kits available from TAKARA Co., Ltd.

Once the nucleotide sequence of the gene is determined, then the gene of the present invention can be obtained by chemical synthesis, PCR using a cloned cDNA as a template, or by conducting hybridization using the DNA fragment having the corresponding nucleotide sequence as a probe.

The obtained KIAA0172 gene can be incorporated into a suitable expression vector available and is used to further transform a suitable host cell, which is cultivated in a suitable culture medium to express the gene and to obtain the object protein that can be collected and purified. Any vector such as a plasmid, phage and virus can be used for this purpose as long as it can be reproduced in a host cell. Examples thereof include Escherichia coli plasmids such as pBR322, pBR325, pUC118, pUC119, pKC30, pCFM536, bacillus subtlis plasmids such as pUB110, yeast plasmids such as pG-1, YEp13, YCp50, DNA of phages such as λgt110 and λZAPII, and examples of a vector for mammalian cells include viruse DNA such as baculovirus, vaccinia virus and adenovirus, and SV40 and a derivative thereof. A vector contains replication origin, selection marker and promoter and, if needed, enhancer, transcription termination sequence (terminator), ribosomal binding site, polyadenylated signal and the like.

Examples of the host cell include bacteria cells, such as E. coli, Streptomyces, and Bacillus Subtlis, fungus cells, such as Aspergillus Stlanes, yeast cells, such as baker's yeast and methanol-utilizing yeast, insect cells, such as Drosophila S2 and Spodoptera Sf9 cells, and mammalian cells, such as CHO, COS, BHK, 3T3, and C127.

Transformation can be performed by any known methods such as calcium chloride-mediated transfection, calcium phosphate-mediated transfection, DEAE-dextran-mediated transfection, and electroporation.

The resultant recombinant protein can be separated and purified by various types of separation and/or purification methods. For example, ammonium sulfate precipitation, gel filtration, ion exchange chromatography, affinity chromatography, etc. can be used singly or in a suitable combination.

The amino acid sequence of the protein encoded by the KIAA0172 gene is exemplified in SEQ ID No. 1. The protein, however, may contain or be subjected to mutations such as deletion, replacement and addition of multiple, or preferably a couple of, amino acids in the amino acid sequence as long as the protein containing this amino acid sequence has function equivalent to the activity of the protein encoded by the KIAA0172 gene. One to 10, preferably 1 to 5, and most preferably 1 or 2 amino acids may be deleted from the amino acid sequence represented by SEQ ID No. 1, and 1 to 10, preferably 1 to 5, and most preferably 1 or 2 amino acids may be substituted with another amino acid in the amino acid sequence represented by SEQ ID No. 1. In addition, 1 to 10, preferably 1 to 5, and most preferably 1 or 2 amino acids may be added to the amino acid sequence represented by SEQ ID No. 1. The function of the protein encoded by the KIAA0172 gene as used herein means the function which suppresses uncontrolled proliferation observed in cancer cells. Whether the gene has such function or not can be determined by, for example, introducing a KIAAO 172 gene into an established renal cancer cell line such as HEK293 cell, and observing decrease in cell division frequency per unit time and/or change in the cell morphology such as an increase in the adhesion area of the cell.

Therefore, genes which encode a protein containing the amino acid sequence represented by SEQ ID No. 1, a protein containing an amino acid sequence represented by SEQ ID No. 1 in which one or more amino acids are deleted, substituted or added, or a protein having the function of the protein derived from KIAA0172 gene can also be used for the present invention.

A polypeptide encoded by a mutated KIAA0172 gene found in a cancer tissue and having a changed amino acid sequence can also be used for the present invention. Such mutated nucleotide found in a cancer tissue include a change in the 52nd codon from CAC to CAG resulting in a change of amino acid from His to Gln; a change in the 168th codon from GCG to GTG resulting in a change of amino acid from Ala to Val; an insertion of GCTGTA between the 268th and 269th codons resulting in an insertion of Ala-Val; a change in the 269th codon from GTA to GGA resulting in a change of amino acid from Val to Gly; a change in the 274th codon from GAG to CAG resulting in a change of amino acid from Glu to Gln; a change in the 306th codon from TCC to GCC resulting in a change of amino acid from Ser to Ala; a change in the 506th codon from GCA to GTA resulting in a change of amino acid from Ala to Vla; a change in the 509th codon from CGT to CAT resulting in a change of amino acid from Arg to His as shown in FIG. 6.

The possibility of being affected by cancer or the risk to be affected by cancer can be evaluated by detecting these mutations in the KIAA0172 gene. Furthermore, the possibility of being affected by cancer or the risk to be affected by cancer can be evaluated also by detecting single nucleotide polymorphisms (SNPs) in the KIAA0172 gene without mutation of the amino acid shown below.

The single nucleotide polymorphisms in KIAA0172 gene are as follows. As used herein, the term T/G polymorphism site means that a nucleotide T in the wild type has changed to a nucleotide G in the mutated type.

(i) T/G polymorphism site for the third nucleotide of the codon number 273;

(j) G/C polymorphism site for the third nucleotide of the codon number 299;

(k) C/T polymorphism site for the 1st nucleotide of the codon number 372;

(l) T/G polymorphism site for the third nucleotide of the codon number 380;

(m) T/G polymorphism site for the third nucleotide of the codon number 497;

(n) C/T polymorphism site for the third nucleotide of the codon number 453;

(o) C/T polymorphism site for the third nucleotide of the codon number 478;

(p) G/T polymorphism site for the third nucleotide of the codon number 507;

(q) C/T polymorphism site for the third nucleotide of the codon number 1003; and

(r) G/C polymorphism site for the third nucleotide of the codon number 1120.

In the case that one or more, preferably two or more and most preferably all of these single nucleotide polymorphism sites are mutated, it can be judged that the risk to be affected by cancer is high.

In consideration of the frequency of mutation as shown in FIGS. 6 and 7, detection of mutations and/or single nucleotide polymorphisms of a change in the 274th codon from GAG to CAG resulting in a change of amino acid from Glu to Gln; a change in the 306th codon from TCC to GCC resulting in a change of amino acid from Ser to Ala; a change in the 509th codon from CGT to CAT resulting in a change of amino acid from Arg to His; and G/C polymorphism of the third nucleotide of the codon number 299; CIT polymorphism of the third nucleotide of the codon number 453; C/T polymorphism of the third nucleotide of the codon number 478 are associated with cancer and detection of these mutations and/or single nucleotide polymorphisms is useful for diagnosis of cancer or the evaluation of the risk to be affected by cancer.

Mutations and single nucleotide polymorphisms can be detectable by PCR method, Southern hybridization method, Northern hybridization method, quantitative PCR method, in situ hybridization method, FISH (Fluorescence In Situ Hybridization), PCR-RFLP method, PCR-SSCP method, etc. using the gene of the present invention, a fragment thereof or a complementary DNA thereof. When these mutations and single nucleotide polymorphisms are detected, existence of the mutation in the DNA is directly detectable. Alternatively, absence of mutations and single nucleotide polymorphisms may be detected.

For example, a probe complementary to the nucleotide sequence containing a mutated nucleotide or single nucleotide polymorphism site in the KIAA0172 gene which has a mutation or single nucleotide polymorphism and a probe complementary to the nucleotide sequence in the wild type gene containing the portion corresponding to this mutated nucleotide sites are prepared first. Although the length of the probe to be used is not limited and the full length of the nucleic acid fragment to be amplified by the below-mentioned nucleic acid amplifying method may be used, 15bp to 100bp is usually preferable, and 15bp to 50bp is more preferable and 18bp to 30bp is particularly preferable. As for probes, those labeled with a radioisotope, a fluorescent substance, an enzyme, etc. can be used. Subsequently, gene fragments containing the mutated nucleotide sites in the sample are amplified by the nucleic acid amplifying method, and this amplified fragment and the probe are allowed to react. Whether the KIAA0172 gene has a mutation or a single nucleotide polymorphism can be determined by investigating with which probe the sample DNA hybridizes among probes corresponding to the wild type, a mutant and a single nucleotide polymorph.

Hybridization conditions for detecting a mutation or single nucleotide polymorphism using a probe can be set up suitably. The hybridization condition that enables only a single-nucleotide mismatch to be detected can be selected by adjusting the temperature and salt concentration at the time of hybridization. Specifically, for example, the hybridization can be performed under a condition where the sodium concentration is 150 to 900 mM, preferably 600 to 900 mM, and the temperature is from 60 to 68° C., preferably at 65° C., although depending on the length of the probe DNA to be used.

A fragment of the KIAA0 172 gene or a DNA complementary thereto can also be used as a primer. Sequences complementary to the ends of the region to be amplified between which is located a mutated site or single nucleotide polymorphism site in the KIAA0 172 gene can also be used as primers used for nucleic acid amplification. Although the sequence length of the region to be amplified is not limited, it can be several tens to several hundreds nucleotide. The sequence length to be amplified may be set up so that only one mutation or single nucleotide polymorphism site in the KIAA0172 gene is contained therein or two or more sites of mutation or single nucleotide polymorphism are contained therein. It is also possible to set the primer corresponding to the region containing a mutation site. There is no restriction in the length of primer but it is preferably 15 bp to 50 bp, more preferably 20 bp to 30bp.

Furthermore, a DNA chip for determining the risk to be affected by cancer can be produced using the KIAA0172 gene or a fragment thereof or a DNA complementary thereto. Fragments to be bound to a DNA chip include fragments containing the above-mentioned mutated site or single nucleotide polymorphism site.

Furthermore, the present invention includes not only DNA containing the KIAA0172 gene sequence but also RNA which encode the said gene sequence as well as such DNA or RNA modified. The term modified as used herein include KIAA0172 nucleotide sequences in which certain nucleotides are modified to an extent not to lose the function of the KIAA0172 gene.

The antibody against the protein or polypeptide (the terms protein and polypeptide are not distinguished in this specification) encoded by the KIAA0172 gene can be obtained by immunizing an animal with an expression product of the KIAA0172 gene according to a normal method. An antibody includes a polyclonal antibody and a monoclonal antibody.

A DNA or RNA nucleotide containing the KIAA0172 gene sequence of the present invention can be used for therapeutic treatment of renal cancer or cancer in the other tissues using the technology of gene therapy. For example, the protein encoded by the KIAA0 172 gene can be expressed in a cancer cell in situ to suppress the proliferation of the cancer cell. For this purpose, DNAs or RNAs containing the KIAA0172 gene sequence can be introduced using a vector which is used for gene therapies such as an adenovirus vector, an adeno-associated virus vector, a herpes virus vector, a retrovirus vector, and a lentivirus vector. Such DNAs or RNAs can be directly introduced by injection or gene gun method. Administration may be conducted orally, by injection, or by any administration method as long as it allows DNA to be introduced into the body. Furthermore, the KIAA0172 gene or a vector containing the KIAA0172 gene may be directly administrated into the body (in vivo approach) or incorporated into a cancer cell after it has been taken out of the body followed by returning to the body (ex vivo approach).

The antisense DNA and antisense RNA of the KIAA0172 gene can also be used for treating cancer. Examples thereof include application of the antisense DNA or antisense RNA to the variant of the KIAA0172 gene found in the cancer tissue (FIG. 6).

A protein encoded by the KIAA0172 gene or a part thereof can also be administrated to a patient with cancer in order to suppress the proliferation of the cancer cell thereby treating the cancer.

The therapeutic agent comprising DNA or RNA containing the KIAA0172 gene sequence or a protein encoded by the KIAA0172 gene may contain a pharmaceutically acceptable carrier. Water, sugars such as sucrose, sorbitol and fructose, glycols such as polyethylene glycol and polypropylene glycol, oils such as sesame oil, olive oil and soybean oil, and antiseptics such as p-hydroxybenzoic acid ester, etc. can be used as a carrier for an oral liquid preparation such as a suspension and syrup. Therapeutic agents in the form of powder, a pill, a capsule, and a tablet may contain excipients such as lactose, glucose, sucrose, mannitol, disintegrating agents such as starch and sodium alginate, lubricants such as magnesium stearate and talc, binders such as polyvinyl alcohol, hydroxypropylcellulose and gelatin, surfactants such as fatty acid esters and plasticizers such as glycerin esters, etc. In addition, a solution for an injection agent can be prepared using a carrier which consists of distilled water, salt solution, glucose solution, etc. For this purpose, it is prepared as a solution, suspension or dispersion using a suitable solubilizing agent and a suspending agent according to a conventional method.

Furthermore, cancer can be detected by either qualitative or quantitative tests of the existence, expression, and mutation of the KIAA0172 gene in each tissue by Northern hybridization method, PCR method, quantitative PCR method, RT-PCR method, in situ hybridization method, etc. utilizing DNA or RNA nucleotide containing the KIAA0172 gene sequence. A part of the KIAA0172 gene sequence can also be used as a primer of PCR or a probe for detection. These polynucleotides are labeled by nick translation etc. In a practical application, expression of the KIAA0172 gene can be examined by RT-PCR method to detect cancer or the degree of progression of cancer. Existence of the KIAA0172 gene DNA can be examined by PCR or quantitative PCR. Cancer can also be detected by detecting DNA or RNA containing the KIAA0172 gene sequence using a tissue section, a cell, a chromosome, etc. by the in situ hybridization method using a DNA or RNA probe containing a part or whole of the KIAA0172 gene sequence. For this purpose, a cancer can be directly detected by investigating the existence of the said mutated gene using DNA and RNA containing a mutated KIAA0172 gene sequence found in a cancer tissue (FIG. 6) or a part thereof. Furthermore, it is also possible by investigating the amount of transcription/expression of the KIAA072 gene by RT-PCR to detect cancer. If there is little or no transcription and expression of the KIAA0172 gene detected, it will be judged that the possibility of carcinogenesis is high.

An antibody which recognizes a polypeptide encoded by the KIAA0172 gene can also be used for detection of cancer. For example, the expression product of the KIAA0172 gene can be measured by immunoassay technology such as EIA and RIA using an antibody which recognizes the protein encoded by the KIAA0172 gene to detect cancer. Alternatively, a section of the tissue may be prepared and immuno-stained using the antibody. Immunostaining can be performed by a state-of-the-art method. In this case, when the protein encoded by the KIAA0172 gene is not detected, it will be judged that there is a possibility of carcinogenesis.

In the present context, an antibody which specifically recognizes a protein having a mutated amino acid sequence encoded by the mutated gene but not the protein not having a mutated amino acid sequence can also be used to measure the protein produced by the mutated gene thereby detecting cancer.

These detections can be performed by contacting antibodies against the protein encoded by the KIAA0172 gene or DNA containing the KIAA0172 gene, etc. with the sample such as body fluid, a piece of the tissue, cells and chromosomes obtained from the subject for which the diagnosis of cancer is conducted. Moreover, it is also possible to administrate these DNAs and antibodies into a patient for the detection.

A detection kit containing an antibody against the protein from the KIAA0172 gene and a nucleotide containing the KIAA0172 gene sequence can be also produced. For this purpose, it is preferable to contain a reference material for quantification in the kit.

Furthermore, the risk to be affected by cancer can be evaluated by analyzing the loss of heterozygosity (LOH) in the genome region containing the KIAA0172 gene. The KIAA0172 gene exists near microsatellite markers D9S1779 and D9S1858 on chromosome 9p24, and evaluation can be effected just only by specifying LOH using these two microsatellite markers. When LOH is specified, it can be estimated the higher risk of carcinogenesis.

Specification of LOH can be conducted by, for example, gene scan analysis, RFLP method using Southern blot method, PCR-RFLP method, and single-stranded DNA high order structure polymorphism analyzing method [PCR to SSCP (single-stranded conformation polymorphism)] using PCR method (for detailed methods, see ‘the basic technology of genetic engineering’, in Biotechnology Manual Series 1, ed. by Masashi Yamamoto, Yodosha Co., Ltd. (1993)).

For example, when analyzing LOH by SSCP method, genes in the vicinity of containing the gene polymorphism site to be analyzed are amplified using the PCR method, resultant PCR products are denatured to be single strands and then subjected to non-denaturing polyacrylamide gel electrophoresis. Differences in the sequence can be analyzed as changes in the mobility due to the differences in the high order structure of the single-stranded DNAs. Consequently, when the resulted peaks or bands of DNAs representing the number of alleles agree with each other between analyte and/or normal cells, the individual is judged as “heterozygous” in the gene polymorphisms; however, when the signal-intensity balance between paired peaks or bands of DNAs originating from the gene analyte and normal cells is lost, it is regarded that a loss of heterozygosity occurs, and the case is judged as LOH.

Since the inactivation of the KIAA0172 gene relies on methylation, the risk to be affected by cancer can be evaluated also by analyzing the methylation pattern of the KIAA0172 gene. Though, the methylation analysis can be carried out in any region within the KIAA0172 gene, it may be conducted, for example, for the CpG island existing in exon 1. The larger the degree of methylation, the larger the risk to be affected by cancer. Analysis of methylation can be achieved by methylation-specific PCR. The technique of methylation-specific PCR is disclosed in Proc. Natl. Acad. Sci. USA, 1996, 93:p.9821-9826, by Herman, J. G. et al. The length of a primer to be used for this purpose is preferably at least 20b, and containing 12 g or c in total. As long as the primer length is 50b or less, it may be longer than the PCR primer; which is usually 20 to 25b.

### BRIEF DESCRIPTION OF THE DRAWINGS

FIG. 1 shows the genome structure of the KIAA0172 gene;

FIG. 2 shows the structure of the KIAA0172 gene. FIG. 2A shows structure of the KIAA0172mRNA, FIG. 2B shows the cap site, FIG. 2C shows the result of electrophoresis for determining the sequence nucleotide of the cap site and FIG. 2D shows the sequence nucleotide at the start of the gene structure;

FIG. 3 shows the structure (amino-acid sequence) of the KIAA0172 gene. The portion in a box containing the 1006th to 1162nd nucleotide indicates the ankyrin homologous site;

FIG. 4 shows the result of LOH analysis of renal cancer;

FIG. 5 shows the gene expression state in renal cancer patients by RT-PCR;

FIG. 6 shows the gene mutation in renal cancer;

FIG. 7 shows single nucleotide polymorphisms (SNPs) on the KIAA0172 gene;

FIG. 8 shows the relation among LOH, mutation of the sequence nucleotide and SNP;

FIG. 9 is a picture showing the intracellular localization of the KIAA0 172 gene;

FIG. 10 shows the relation between intracellular localization and existence of protein of the KIAA0172 gene. FIG. 10A is a picture showing the results of immunostaining experiment using an anti-KIAA0172 protein antibody, and FIG. 10B shows the results of immunoprecipitation and western analysis using an anti-KIAA0172 protein antibody;

FIG. 11 is a picture showing the results of immunostaining of normal and cancer tissues using an anti-KIAA0172 protein antibody;

FIG. 12 shows allele-specific gene expression of the KIAA0172 gene. FIG. 12A shows the allele loss result in the cancer tissue DNA in gene scan analysis, FIG. 12 B shows the gene expression loss result in the cancer tissue by RT-PCR method and FIG. 12C shows the allele-specific expression result using single nucleotide polymorphism;

FIG. 13 shows methylation pattern of the KIAA0172 gene in the normal and cancer tissues and an established cancer cell;

FIG. 14 is a picture showing an activation of the gene expression of the KIAA0 172 gene by the treatment of 5-aza-2′-deoxycytidine;

FIG. 15 shows proliferation suppressing ability of the KIAA0172 gene in the colony formation experiment using HEK293 cell. FIG. 15 A shows expression in HEK293 cell and FIG. 15 B shows cell proliferation suppressing ability when transfected into HEK293 cell;

FIG. 16 shows proliferation suppressing ability of the KIAA0172 gene in the colony formation experiment using G-402 cell. FIG. 16 A shows the result of RT-PCR indicating that no expression of the KIAA0172 gene was observed in G-402 cell and FIG. 16 B shows cell proliferation suppressing ability when transfected into G-402 cell;

FIG. 17 is a picture showing the transformation ability for HEK293 renal cancer cell line derived from renal cancer; and

FIG. 18 shows proliferation suppressing ability of the KIAA0 172 gene observed in the cell proliferation suppressing experiment using a nude mouse. FIG. 18 A is a picture showing the formation of cancer in a nude mouse into which cells having introduced a plasmid (pCMV-KIAA) or only an empty vector were introduced and FIG. 18 B is a graph showing the formation of cancer.

### BEST MODE FOR CARRYING OUT THE INVENTION

The present invention will be described specifically by way of Examples below. However, the technical scope of the present invention is not limited by these examples.

### EXAMPLE 1

### Structural Analysis of the KIAA0172 Gene

(1) Genome Structure of the KIAA0172 Gene

This gene is located on BAC clone RPCI-11-130C19 mapped to chromosome 9p24 existing between the nucleotide numbers 85563-120956 on this clone. EST was reported as WI-12779, and the cDNA nucleotide sequence of almost full length was reported in the following paper.

Nagase, T., Seki, N., Ishikawa, K., Tanaka, A. and Nomura, N. “Prediction of the coding sequences of unidentified human genes. V. The coding sequences of 40 new genes (KIAA0161-KIAA0200) deduced by analysis of cDNA clones from human cell line KG-I.” DNA Res. 3 (1), 17-24 (1996).

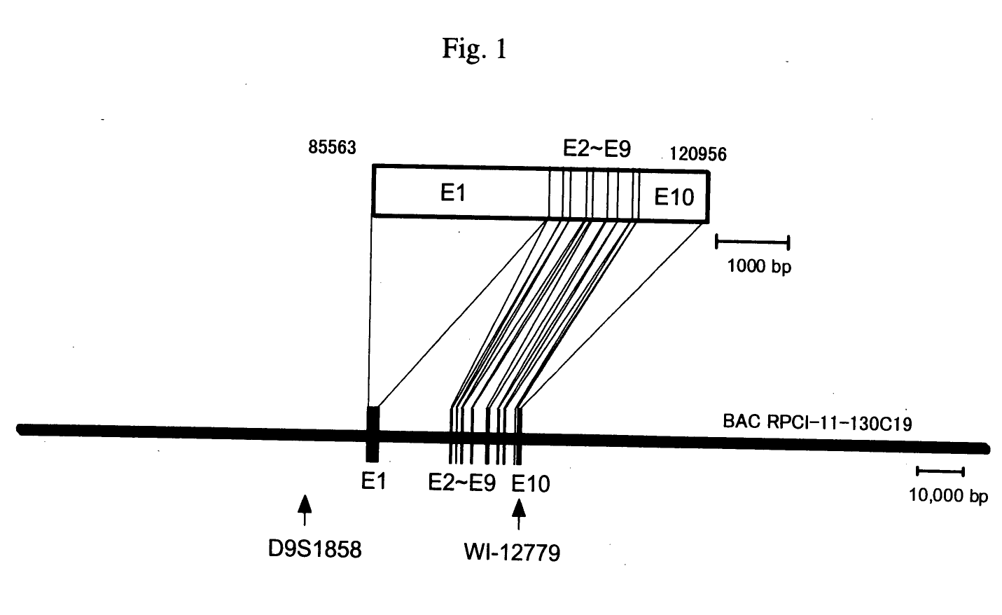

A part of cDNA sequence was also reported to GenBank (1996) under accession number D79994 (registered as : Human mRNA for KIAA0172 gene, partial cds). The comparison between the above-mentioned BAC clone and the said paper and the result of Example (2) revealed that the gene has a structure consisting of 10 exons as a result of the present inventors’ analysis (FIG. 1). The drawing shows the structure of KIAA0172 gene on the human genome. There are ten exons in the range of nucleotides 85563-120956 on BAC (Bacterial Artificial Chromosome) RPCI-11-130C19. The position of microsatellite marker D9S1858 and EST(Expressed Sequence Tag) WI-12779 are also shown.

(2) Structure of the KIAA0172 Gene

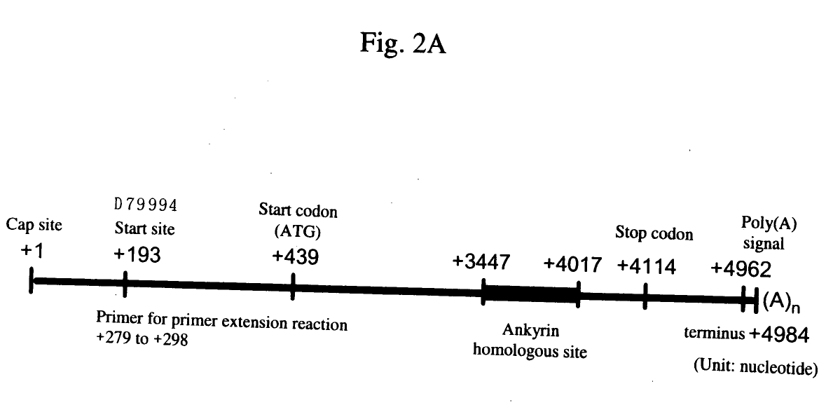

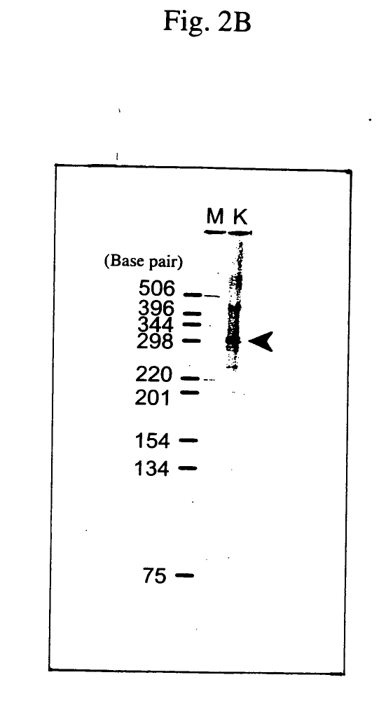

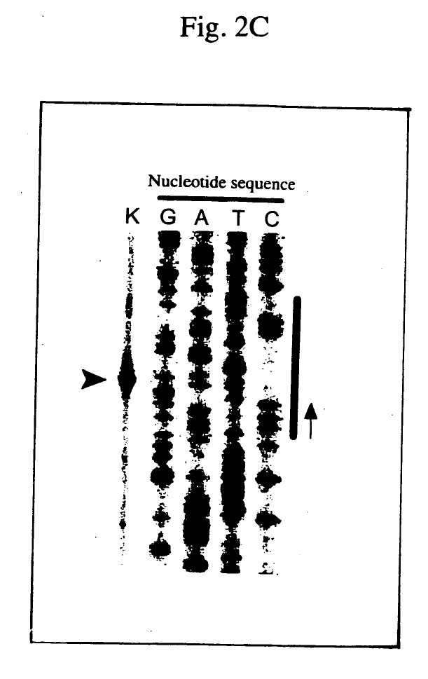

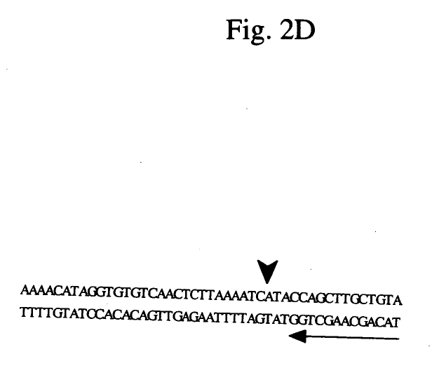

Since the above-mentioned paper and the data in GenBank database does not determined the full length of cDNA, the perfect amino acid sequence of the protein of the gene product has not been decided. We performed the primer extension reaction in order to determine whole gene structure and decided the cap site which is 5′ end of the gene. The sequence of primer used in extension reaction was CAGATGTGGTCCTGGGTTCT (SEQ ID No. 36) which is the antisense strand downstream after the 87th base pair on the KIAA0172 cDNA nucleotide sequence obtained from the database. The primer labeled with ³²P using T4 polynucleotide kinase was allowed to anneal with 10 μg of RNA derived from human kidney in a 30 μl of 40 mM PIPES buffer containing 80% formamide at 45° C. for 12 hours. The Reaction product was purified and submitted for further reaction using 100 units of M-MuLV reverse transcriptase (New England Biolabs) in a reverse transcription reaction mix which contains 20 μl of 0.5 mM dNTPs at 37° C. for 2 hours. The product was separated and analyzed using 6% polyacrylamide—7 M urea gel. FIG. 2 shows the gene structure of the KIAA0172 gene. Although the partial cDNA structure of this gene was already reported, complete cDNA structure (and amino acid sequence of the whole gene product) has been now determined by determining the cap site which has been left unreported (FIG. 2A). The extension reaction was began from the primer site shown in the drawing using the primer extension method, the cap site was identified from the position of the band on gel (FIG. 2B), and the nucleotide sequence of cap site was determined (FIG. 2C). The arrowed band in lane K shows the primer extension reaction product in FIG. 2B, and lane M shows a size marker. Consequently, it became clear that the gene begins from the nucleotide sequence shown in FIG. 2D. Thus the gene structure was completely determined.

The nucleotide number 85563 on the BAC clone RPCI-11-130C19 was identified as a cap site. Index number “+1″ of the KIAA0172 gene (mRNA) was assigned to this position, and the corresponding nucleotide numbering was given thereby. The index number +439 is the start codon (methionine), and an ankyrin homologous site considered to be the functional site is located from +3447 to +4017, stop codon at +4114 and the poly (A) signal at +4962. The total length was 4984 nucleotide (FIG. 2). The exon structures (the nucleotide number on BAC clone RPCI-11-130C19 and the length of each exon) and the positions of cap site, the start codon, the poly(A) signal, and the poly (A) addition (all are the nucleotide numbers on BAC clone RPCI-11-130C19) are as follows (FIG. 1). Exon 1: 85563-88317 (2755 base pairs) Exon 2: 104904-105101 (198 base pairs)

Exon 3: 106011-106119 (109 base pairs)

Exon 4: 107231-107470 (240 base pairs)

Exon 5: 109601-109688 (88 base pairs)

Exon 6: 113138-113357 (220 base pairs)

Exon 7: 115645-115787 (143 base pairs)

Exon 8: 117058-117258 (201 base pairs)

Exon 9: 119344-119442 (99 base pairs)

Exon 10: 120026-120956 (931 base pairs)

Total 4984 base pairs

Position of the cap site: 85563 (the same as start position of exon 1)

Start codon position: 86094

Poly(A) signal position: 120936

Poly(A) addition position: 120956 (the same as end position of exon 10)

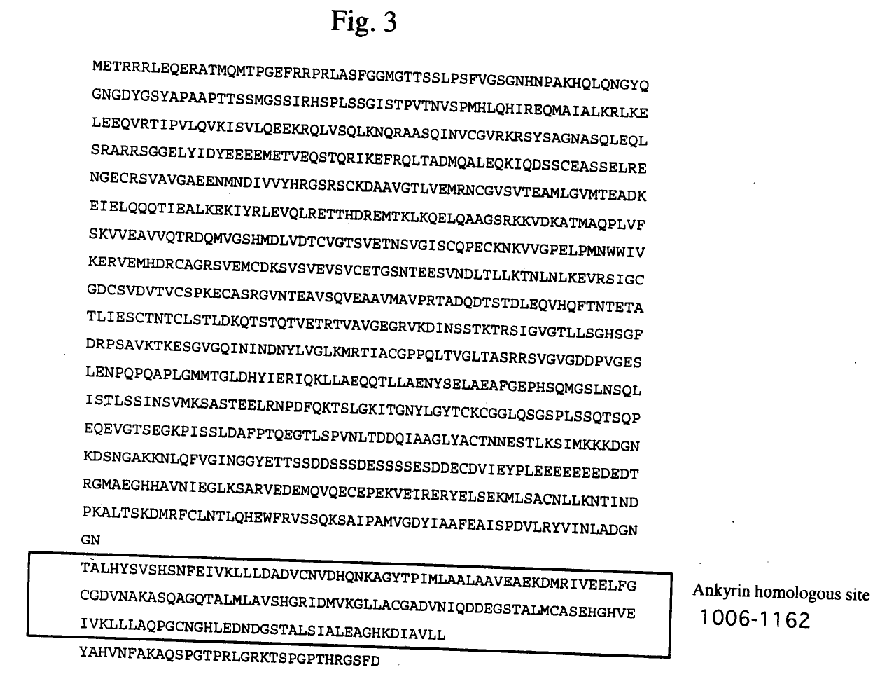

FIG. 3 shows the structure of the protein which is the product of the KIAA0 1 72 gene. The protein consists of 1194 amino-acid residues and has an ankyrin homologous site from 1006th to 1162nd amino-acid residue. It is possible to use such a feature for the production of an antibody, etc.

### EXAMPLE 2

### Functional Analysis of KIAA0172 Gene

(1) Relevance to the Formation of Renal Cancer (part 1)

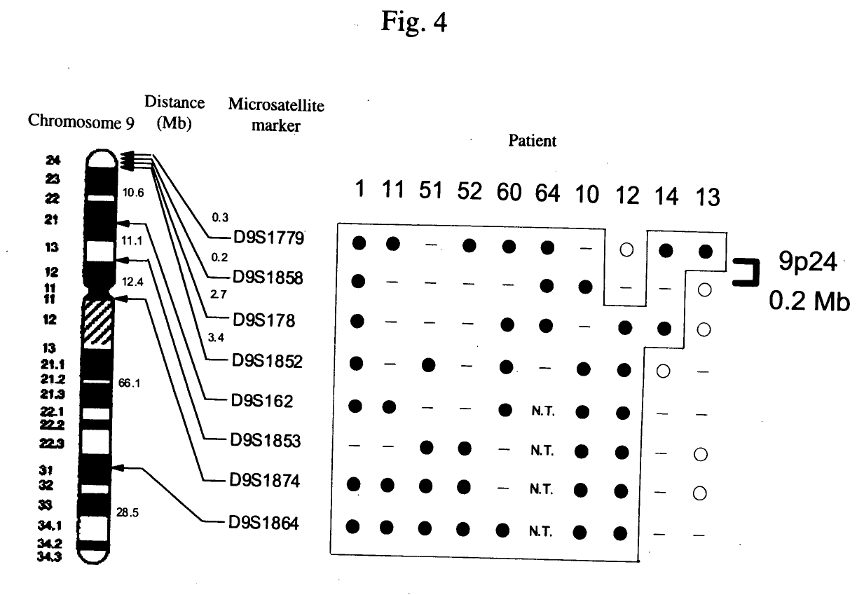

LOH (Loss of Heterozygosity) analysis was performed. LOH was searched in comparison with DNAs obtained from normal and cancer tissue of the renal cancer patient using the microsatellite markers (shown in FIG. 4). The information of the microsatellite markers was obtained from the database HYPERLINK, “http://gdbwww.gdb.org”.

After frozen tissue was digested by Proteinase K, the genome DNA was extracted with phenol/chloroform. The renal cancer tissue used was a granule type or a clear cell type. All the microsatellite markers used was those according to the Stanford University human genome center database. Either one of the primers of sense and antisense strands was labeled with 6-FAM phosphoamidite and used for PCR. PCR was performed using 50 ng of template DNA in 15 μl of the total quantity and GeneAmp PCR system 9700 (Perkin-Elmer Applied Biosystems). The reaction was 25 cycles of 94° C. for 30 seconds, 55° C. for 30 seconds and 72° C. for 1 minute. The fluorescence-labeled PCR product was subjected to electrophoresis and subsequently analyzed using GeneScan 3.1 software. The signal intensity of the normal tissue origin DNA and the tumor origin DNA was compared, and when the intensity was reduced by 33% or more, it was judged as LOH. The genome DNA from the ordinary tissue and that extracted from the primary cells in culture from the tumor tissue were used for LOH analysis of patient R6.

LOH assay was conducted by comparing 49 DNAs from the cancer tissue and corresponding DNA analytes from normal kidney tissues obtained from renal cancer patients using the GeneScan method of ABI. Six and four samples (nine samples out of 49 in total) significantly showed LOH at microsatellite marker sites D9S 1779 and D9S1858 respectively in the vicinity of the gene of interest (within less than about 2OOkbp). Among these, two samples indicated LOH at only one microsatellite marker site. The minimum common deleted region determined by comparing all the samples exhibiting LOH was allocated into the 165 kb region between the above-mentioned two microsatellites.

The above-mentioned result shows that the rate of mutation in the gene region concerned is at least about 18%, which is significantly high as a rate of mutation. Moreover, the fact that the common deleted region exists near the gene concerned suggests the involvement of the gene in the renal cancer formation. FIG. 4 shows results of LOH analysis testing relationship between KIAA0172 gene and renal carcinogenesis. FIG. 4 demonstrates imvolvement of the KIAA0172 gene in the renal cancer formation. The genome DNA originating from a renal cancer patient was used, and the solid circle shows the site which relates to renal cancer in the LOH (Loss of Heterozygosity) analysis (gene scan analysis) using the microsatellite shown in the drawing, and the open circle shows the site which does not relate to renal cancer. The bar shows the site from which information was not obtained. As a result of this analysis, the 0.2 Mb (mega base pair) region at 9p24 site showed relevance with renal carcinogenesis, and since this gene existed in this site, the imvolvement of this gene was suggested. The results also suggested that the deletion tests at 9p24 site by the present method is possible.

(2) Relevance to the Formation of Renal Cancer (part 2)

The gene expression state in the renal cancer patient was investigated by RT-PCR method. The gene expression states in a normal and cancer tissues derived from 8 renal cancer patients were compared using cDNAs (DNA synthesized by reverse transcription reaction from mRNA) from normal and renal cancer-tissues. Specifically, RT-PCR amplified three types of ESTs, i.e., WI-19184, WI-12779 and WI-17492, which are coded within the candidate region of LOH obtained by LOH analysis and are eventually expressed in the kidney. The reverse transcription reaction was performed by reacting 1 μg of total RNA and 5 pmol oligo(dT) at 37° C. for 1 hour in the presence of 200 units of M-MuLV reverse transcriptase (New England Biolabs). After the reaction was completed, the reaction solution was subjected to a heat treatment at 94° C. for 3 minutes to inactivate the enzyme. PCR reaction solution (15 μl in total) was prepared by addition of 1 μl reverse transcription product and 2.5 pmol specific primers, and the reaction was conducted. The used primer sequences are as follows. For EST WI-17492, forward primer TCAGTCAAGGTCACAGTCATATTAA (SEQ ID No. 37) and reverse primer TTGTGCTGTCTGTCAGCATATG (SEQ ID No. 38); for EST WI-12779, forward primer AAGTAAATGTGACAGGTAAAAAGG (SEQ ID No. 39) and reverse primer CTTGACACAGTATTTTCAGCTTTTG (SEQ ID No. 40); and for EST WI-19184, forward primer GAATTCCTTCCTCCCCTGTC (SEQ ID No. 41) and reverse primer AAACCAGGCACAATCAAACC (SEQ ID No. 42) was used. As for KIAA0172, forward primer GTGGAGACCAGGACAAGGAACAGAAAGAC (SEQ ID No. 43) and reverse primer TCCAGAGGGGGAGGTGGCTTT (SEQ ID No. 44) were used for the 5′-region, the primer set for WI-12779 was used for the 3′-region. The PCR condition was 30 cycles of 94° C. for 30 seconds, 60° C. for 30 seconds and 72° C. for 30 seconds. In compensation of the gene expression levels using RT-PCR between normal and renal cancer tissues, data were normalized using G3PDH gene as an internal control. PCR primer sequences for each of the ESTs were obtained from the HUGO database HYPERLINK “http://gdbwww.gdb.org”.

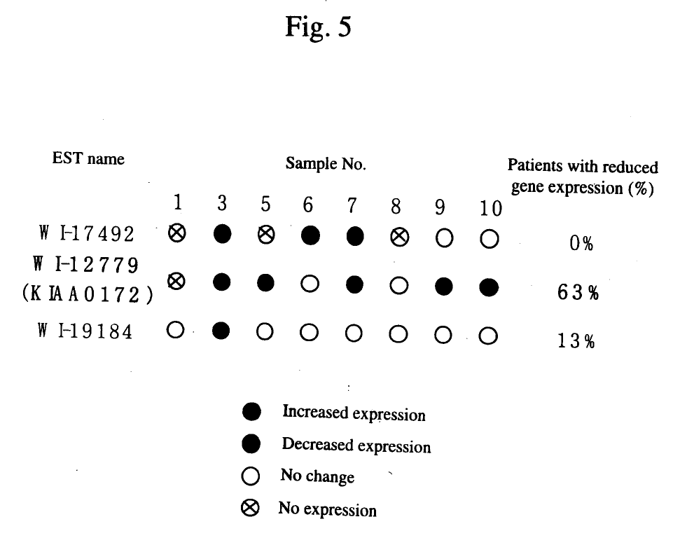

FIG. 5 shows the gene expression state in renal cancer patients by RT-PCR elucidating the relevance of the KIAA0172 gene to the renal cancer formation. The drawing is the result of performing RT-PCR for three types of candidate ESTs (shown in the drawing) using mRNA obtained from normal tissue cell and cancer tissue cell of a renal cancer patients and comparing the amount of gene transcription in the cancer tissue cell against the normal tissue cell. Reduction of the gene expression was observed in about 63% of the patients for WI-12779 among three ESTs as a result of this experiment. Thus, the relevance of this EST to renal cancer was significantly indicated.

The amount of transcription was measured by the same method as the above using two genes (or ESTs) WI-19184 and WI-17492 which exist very near the gene concerned on the human genome, and no significant reduction was observed. Since EST WI-12779 is a part of the KIAA0172 gene, the relevance of the KIAA0172 gene to renal cancer was strongly suggested.

(3) Relevance to the Formation of Renal Cancer (Part 3)

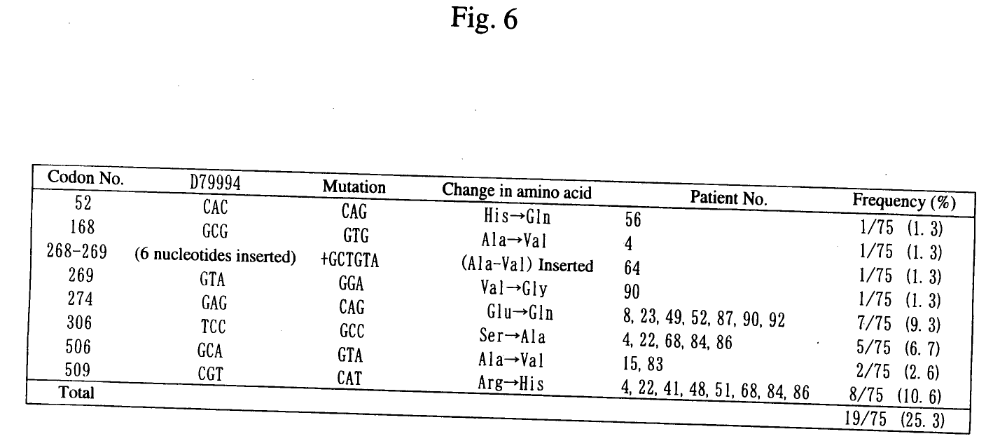

Gene mutation analysis was performed by nucleotide sequencing. Nucleotide sequences were determined for exons 1-10 encoding amino acid sequences. Since exon 1 was long with 2662 bp, it was divided into five parts (a, b, c, d, and e) to determine the nucleotide sequence. PCR was conducted by 35 cycles each consisted of 94° C. for 30 seconds, 60° C. for 30 seconds and 72° C. for 1 minute using 25 ng of DNA templates and 5 pmol of primers. The amplified DNA was subjected to nucleotide sequence determination using ABI Prism 310 Genetic Analyzer. The obtained nucleotide sequence was compared with that of the gene concerned registered into the GenBank database (accession number D79994) and mutated sites were specified (FIG. 6).

The sequences of the primers used are as follows:

EXON 1

(SEQ ID No.2)

E1af: TAC TTT GTG GAG ACC CCC TA

(SEQ ID No.3)

E1ar: GCT TGT GGT GCC CAT GCC TCC

(SEQ ID No.4)

E1ar2: CAC TGG GGT GGA GAT CCC TG

(SEQ ID No.5)

E1bf: ATT ATG GTA GCT ATG CCC CA

(SEQ ID No.6)

E1bt2: TGC AGC ACA TCC GCG AGC AGA T

(SEQ ID No.7)

E1cf: TCC GGC AAC TITA CAG CAG

(SEQ ID No.8)

E1cf2: CAG CTG TGA GGC CTC CTC AG

(SEQ ID No.9)

E1br: GCC TCT GTG GTA CAC GAC GAT G

(SEQ ID No.10)

E1df: AGG CAT CTC CTG CCA GCC TGA AT

(SEQ ID No.11)

E1cr2: TCC ACA GAC CTC CCA GCA CAT C

(SEQ ID No.12)

E1cr: TGT GTG TTG CTG CCT GTT TCG CAG ACG CT

(SEQ ID No.13)

E1dr2: AGA CAA GTG TTG GTG CAG GAC TC

(SEQ ID No.14)

E1ef: GGA CAG TAG CTG TAG GA

(SEQ ID No.15)

E1dr: CAG CTG AT GGC CTG TCA AAC CC

(SEQ ID No.16)

E1er2: GGG TTC CTC AGC TCT TCA GTG C

(SEQ ID No.17)

E1er: TCC TCA TTC CCA GGT CCT CAG G
EXON 2

(SEQ ID No.18)

CAG TCC TAG CAT CAC ACA CTC TG

(SEQ ID No.19)

TCC TGC CAA TGA CTG TGA
EXON 3

(SEQ ID No.20)

GGG TGT GAG TTT TCA TTT TTA TTG CC

(SEQ ID No.21)

ACT GAC AGC ATT AGC CTC TAG AAC
EXON 4

(SEQ ID No.22)

TGA GCA CAC CTT GCA TCT CCT GA

(SEQ ID No.23)

CAT TAA ATG TGG GAG GGG CAA
EXON 5

(SEQ ID No.24)

TCT TCT TGT GAC CAA TCG TAA CTT

(SEQ ID No.25)

TAC ACA CTG GGG ATG GTG TTT GC
EXON 6

(SEQ ID No.26)

AAT AGA AGA ACT AAC GAC CAC TTG G

(SEQ ID No.27)

TTA GAG AAG AGA GGG TGG AAG GG
EXON 7

(SEQ ID No.28)

AGA AGG GGC TGC TTC CTA AGA GA

(SEQ ID No.29)

GGG TGC ATT CCT GAG CAC AGG A
EXON 8

(SEQ ID No.30)

CAG TAC GTA CTT CTG AAG TCC TTG

(SEQ ID No.31)

TCC CAG AGC TCC CGT CCA GAG
EXON 9

(SEQ ID No.32)

GAG AAA CCC AAC ATG GCT TGT TCT

(SEQ ID No.33)

GGG GTC CAC CAG TCT GGT GGA
EXON 10

(SEQ ID No.34)

TGA GGT CAC TTA TTA ACC CCC AGT

(SEQ ID No.35)

GTA TCT GTC ACC CCA ACA GGA AC

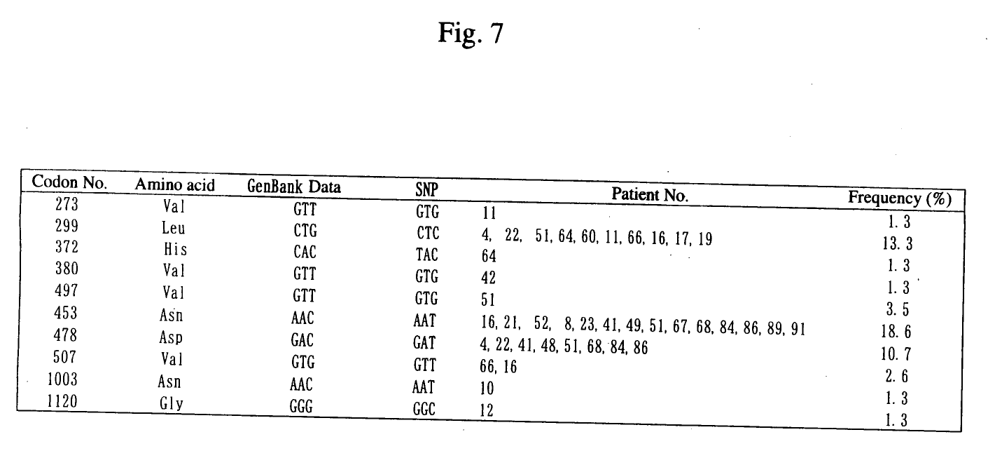

The mutations in the gene concerned were searched by the nucleotide sequencing of genomic DNA obtained from renal cancer tissues of the patients, and the mutation accompanied by change of amino acid sequence (FIGS. 6 and 7) existed in 19 out of 75 samples (25.3%).

FIG. 6 shows the result of the gene mutation analysis by the nucleotide sequencing elucidating the relevance of the KIAA0172 gene to renal cancer formation. FIG. 6 summarizes the amino acid mutation observed in KIAA0172 gene using DNAs from 75 patients with renal cancer. The amino acid sequences (codon number) which are the gene products and the amino acid (sequence of the registration number D79994 in GenBank) already reported were compared. Changes of the amino acid and the number of the patient in which the change was observed and the frequency of the change are indicated.

FIG. 7 shows single nucleotide polymorphisms (SNPs) on the KIAA0172 gene to find a relation with renal cancer formation. FIG. 7 summarizes the result of the investigation on the gene polymorphism the use of which is becoming more and more important in recent years. Polymorphism is the mutation on a gene which does not change amino acid sequence. The drawing shows the codon number and amino acid sequence in which it was observed, the nucleotide sequence registered in GenBank, the nucleotide sequence and the patient number of the single nucleotide polymorphism observed in a patient and the frequency of appearance of polymorphisms.

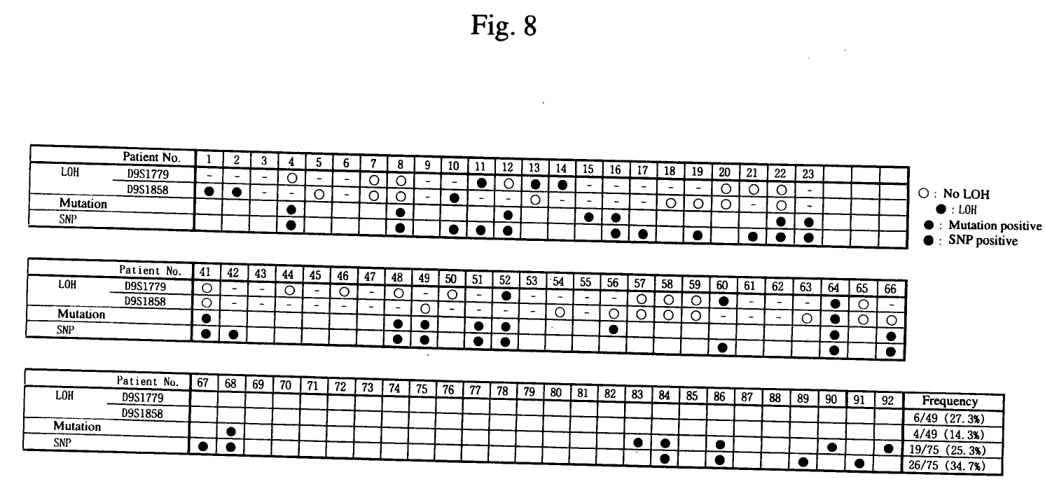

FIG. 8 shows the result of the gene mutation analysis (relation among LOH, mutation and SNP) based on the nucleotide sequencing to prove the relevance of the KIAA0172 gene to renal cancer formation. FIG. 8 summarizes preceding drawings (FIGS. 4, 6, and 7). Consequently, it turns out that there is no particular relation among LOH, mutation and SNP. Therefore, it was confirmed that the decrease in the gene expression of the KIAA0172 is not caused by the gene mutation. This result suggests that the functional analysis of KIAA0172 gene based on information such as LOH, mutation and SNP is possible.

The rate of the gene mutation observed was significantly above the normal rate that is about 1/one million. This high rate of mutation in KIAA0172 gene is unlikely generated irrelevantly to the formation of renal cancer. Moreover, substitutions of the amino acid are not only that with similar amino acid such as alanine to valine, valine to glycine, but also substitution with very different amino acids such as serine to alanine, glutamic acid to glutamine and arginine to histidine that reflects a big functional change in KIAA0172. Furthermore, there was observed an insertion of two amino acid residues. Therefore, the relevance between mutation on KIAA0172 gene and renal carcinogenesis is high.

### EXAMPLE 3

### Functional Analysis of the KIAA0172 Gene

(1) Cell Culture, cDNA and Preparation of Antibody

The renal cancer cells (RCC) were obtained from American Type Culture Collection. VMRC-RCW cells were maintained in a MEM medium and HEK293 cells were maintained in a DMEM medium (both contain 10% Fetal Bovine Serum) at 37° C. in the presence of 5% CO₂. Human RCC tissue and a normal kidney tissue were sampled from surgery specimens and fragmented, and the primary cell culture was performed (Aoyagi, T. et al., Int. J. Urol. 3, 392-396 (1996)). These cells were maintained in DMEM which contains 10% Fetal Bovine Serum. The normal kidney cells maintained the feature of kidney tubule cell. In order to screen ESTs, the pair of cDNA from the normal kidney tissue and cDNA from the tumor of the same patient was purchased from Clontech. In order to prepare a rabbit antibody against the KIAA0172 gene product, cDNA segment corresponding to amino acid 406 to 580 was amplified by PCR and inserted downstream of glutathione S-transferase (GST) gene in a pGEX vector (Pharmacia) with frames aligned to form a fusion protein. The above-mentioned clone was introduced into E. coli cells, induced by IPTG and the resulted protein was purified by glutathione-Sepharose (Pharmacia). This purified protein was used to immunize a rabbit. The immune serum was subjected to affinity purification using a column containing Sepharose bound with an antigen-GST fusion protein.

(2) Determination of Intracellular Localization by Immunostaining Method

The KIAA0172 cDNA was cloned into pcDNA3.1 (+) (Invitrogen). HEK293 cells cultured on a cover glass were transfected with the resulted vector using Lipofectamine 2000 (Invitrogen). On the next day, the cells were fixed with cold methanol (−20° C.). After washed with phosphate buffer solution, the cells were incubated with an antibody against the KIAA0172 gene product at room temperature for 1 hour. The protein was detected under fluorescence microscope (LSM-410, product of Carl Zeiss) by using FITC-labeled rabbit IgG.

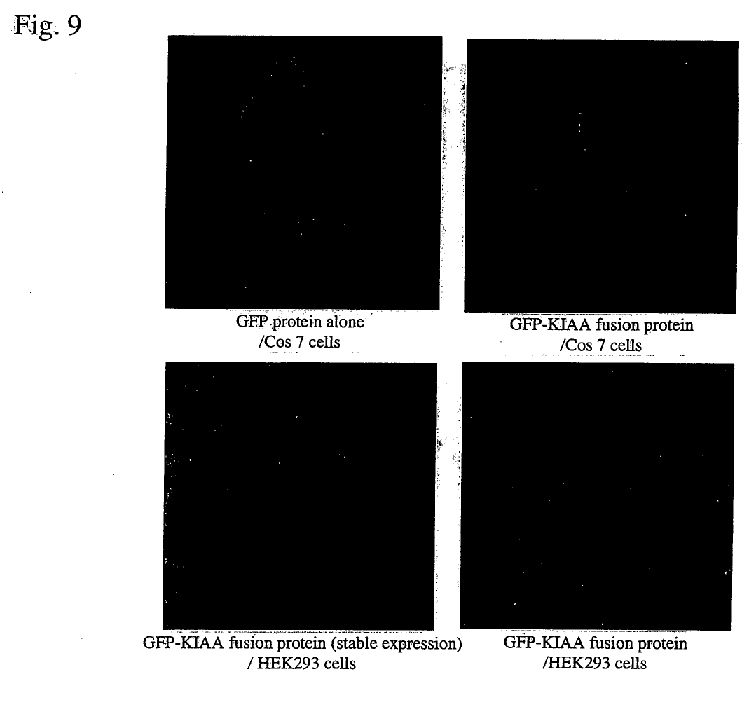

FIG. 9 shows a status of intracellular localization of the KIAA0172 protein. FIG. 9, in which a fusion protein of the KIAA0172 and a GFP (Green Fluorescent Protein) was created and expressed in Cos7 or HEK293 cells. The recombinant protein was readily detected by taking advantage of the fluorescence of the GFP (Green Fluorescent Protein). An image only containing GFP was also shown as a control. This experiment reveals that the protein which is the gene product of this gene is located in cytoplasm. The upper right, upper left, lower right and lower left panels in FIG. 9 indicate expression of GFP-KIAA fusion protein in Cos 7 cells, GFP protein alone in Cos 7 cells, GFP-KIAA fusion protein in HEK293 cells and stable expression of GFP-KIAA fusion protein in HEK293 cells, respectively.

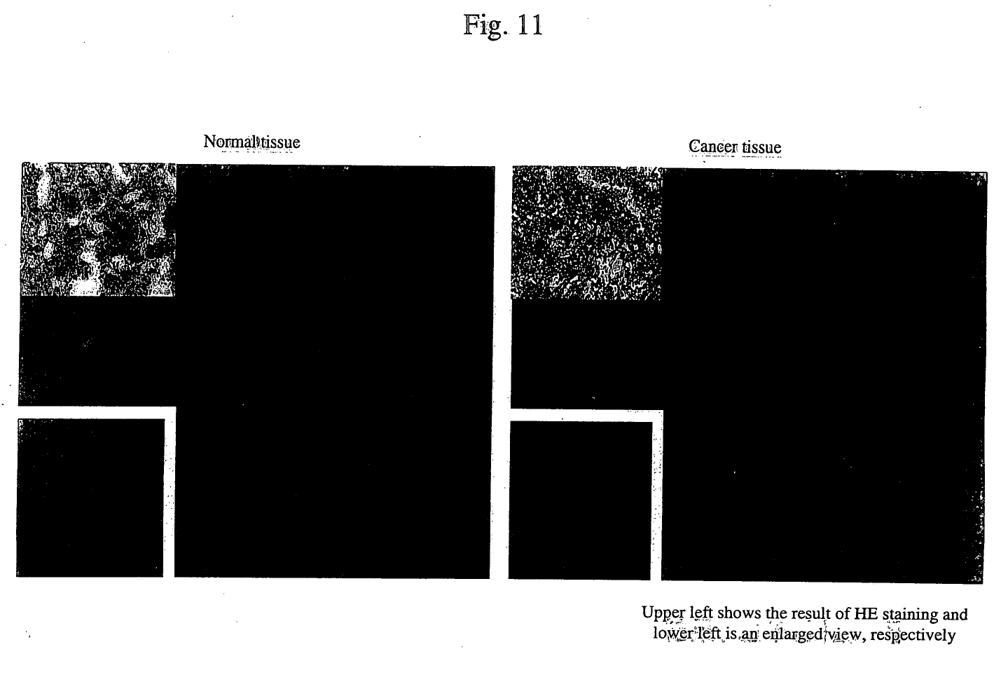

FIG. 11 shows the result of immunostaining of the normal tissue and a cancer tissue using anti-KIAA0172 protein antibody elucidating the function of the KIAA0172 gene. FIG. 11 is an immunostaining image of tissue section of renal cancer and the normal kidney stained by using an anti-KIAA0172 protein antibody (primary antibody). Each tissue was checked by HE staining, and fluorescence detected using a rhodamine-labeled secondary antibody. The left panel shows the result for the normal tissue and the right panel shows the result for the cancer tissue. Insets on the upper left and lower left in each of the panels show the result of HE staining and an enlarged view, respectively. As a result, the protein was detected in the normal tissue, while it was not detected in the cancer tissue. Clinical presentations of the cancer tissue and the diagnosis feasible based on a biopsy using this antibody.

(3) Immunoblotting and Immunoprecipitation

Cell extract was prepared as follows. Cells were washed with phosphate buffer solution three times, and allowed to stand still on ice for 15 minutes in a buffer (50 mM Tris-HCl, pH 7.5, 140 mM NaCl, 10% glycerol, 1% Nonidet P-40, 100 mM NaF, 200 mM NaVO₅, 1 mM PMSF, 10 μg/ml leupeptin, aprotinin and chymotrypsin) to be lysed. The cell lysate was centrifuged at 4° C. for 15 minutes, and used for immunoprecipitation reaction. After the cell lysate and the immunoprecipitate were separated by SDS-polyacrylamide electrophoresis and transferred onto a nitrocellulose membrane. This membrane was subjected to blocking treatment with 5% skim milk, and then allowed to bind with an antibody to the KIAA0172 protein. Alkaline phosphatase-conjugated rabbit IgG (Promega) and BCIP/NBT (GibcoBRL) were used in order to detect the KIAA0172 gene product protein.

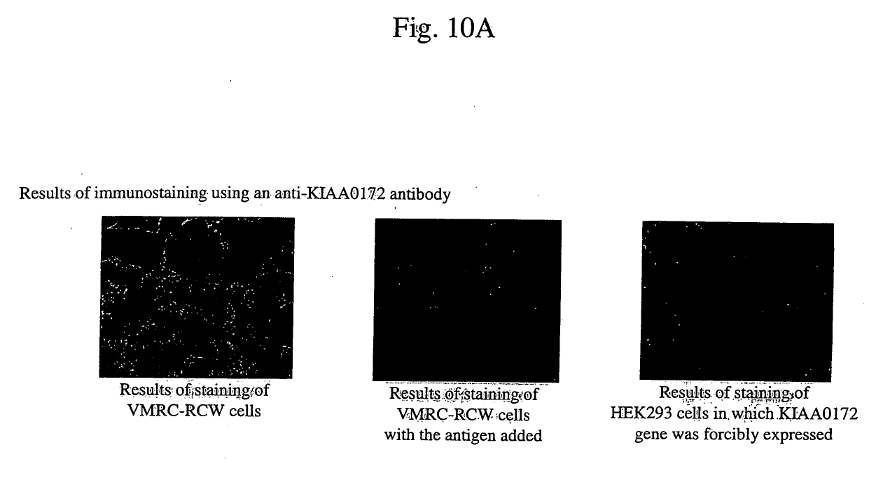

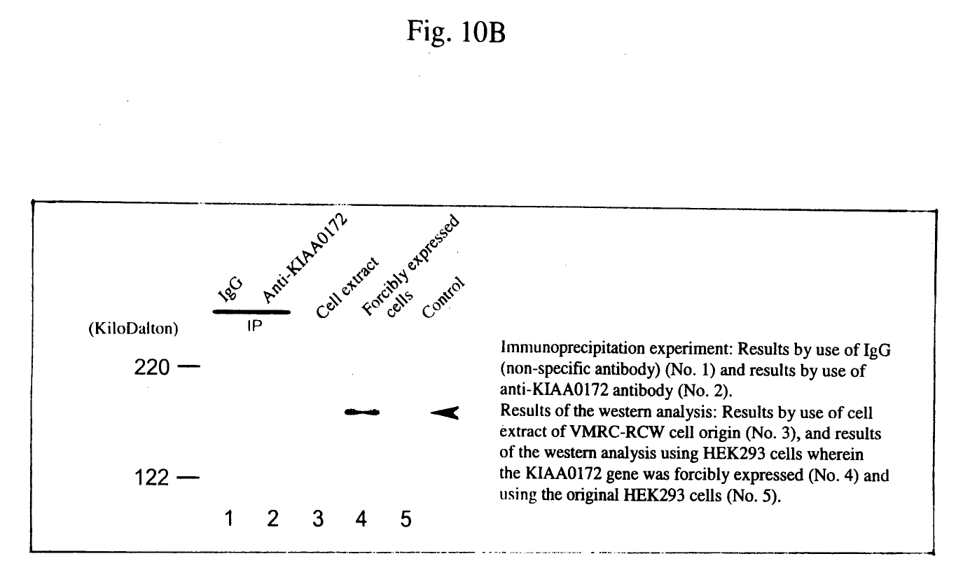

FIG. 10 shows the relation between intracellular localization and existence of protein elucidating the function of the KIAA0172 gene. FIG. 10 showed the results of immunostaining experiment (FIG. 10A) and western analysis (FIG. 10B) against the KIAA0172 gene product using a specific polyclonal antibody (anti-KIAA0172 protein antibody). In the immunostaining experiment VMRC-RCW cells expressing this gene was stained by using the antibody. Two kind of control experiments were performed; the first was an absorption experiment using an antigen treated antibody (where no signal is detected) and the second was an immunostaining experiment using KIAA-null HEK293 cells where the constitutive expression plasmid (pCMV-KIAA) was introduced into to express the gene. In latter experiment, the KIAA0172 protein was found in the cytoplasm. Western analysis using an anti-KIAA0172 protein antibody detects the gene product of the KIAA0 172 in VMRC-RCW cell extract (the antigen-treated IgG did not show a band in a control experiment) and also detects similar bands in an extract of HEK293 cell into which the constitutive expression plasmid (pCMV-KIAA) was introduced. The left panel in FIG. 10A shows the stained result of VMRC-RCW cells, the central panel shows the result of VMRC-RCW cells added with antigens and the right panel shows the stained result of HEK293 cells wherein the KIAA0172 gene was constitutively expressed. In FIG. 10B, the lane number 1 shows the result of immunoprecipitation using IgG, and the lane number 2 shows the result of immunoprecipitation using an anti-KIAA0172 antibody. Number 3 shows the result of the western analysis using an extract of VMRC-RCW cell, number 4 shows the result of the western analysis using HEK293 cells wherein the KIAA0172 gene was constitutively expressed and number 5 shows the result of the western analysis using the original HEK293 cells. Conclusively, the protein which is the product of this gene within a cell has been successfully identified by the immunostaining method and western analysis.

(4) Gene Polymorphism Analysis

The genome DNA (50 μg) from the primary culture cell (R6N) from a normal kidney tissue of the patient R6 was amplified using primer Cf (GCAGCTGTGAGGCCTCCTCAG) (SEQ ID No. 45) and Cr (TCCACAGACCTCCCAGCACATC) (SEQ ID No. 46). PCR was conducted under a condition of 30 cycles of 94° C. for 30 seconds, 60° C. for 30 seconds and 72° C. for 45 seconds. cDNA was prepared from the primary culture cells R6N. PCR amplification was performed on the same conditions as above. The PCR product was purified by spin column (Qiagen) and then sequenced in the both directions by a kit manufactured from Perkin Elmer Applied Biosystems along with Cf and Cr primers. In order to confirm the obtained sequence, the PCR product was cloned into vector pGEM-T (Promega). The resulted clone was selected at random and the sequence was confirmed by the same primers.

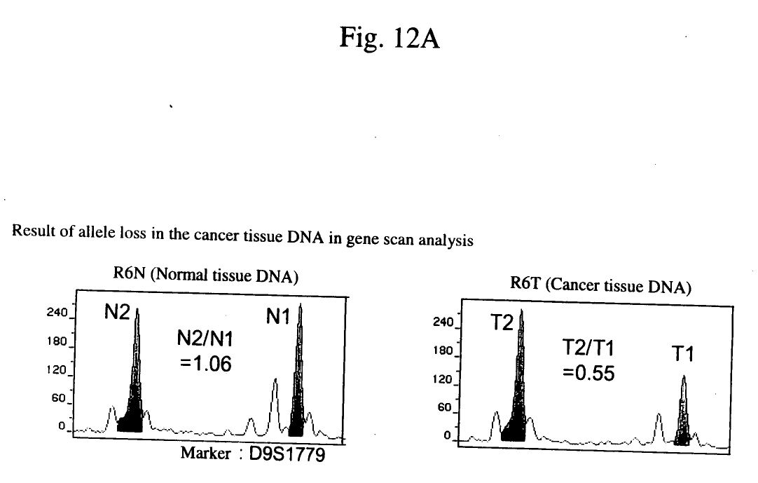

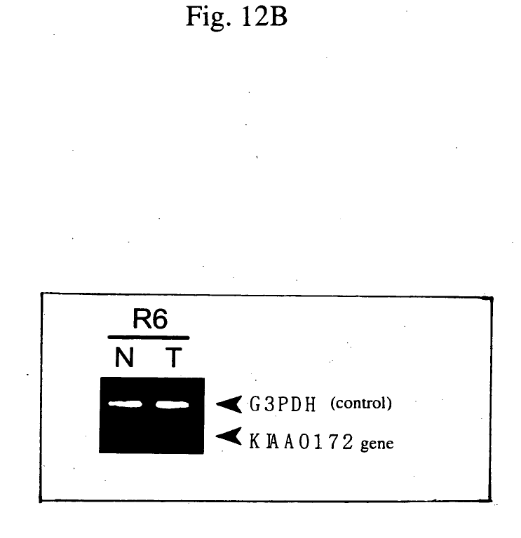

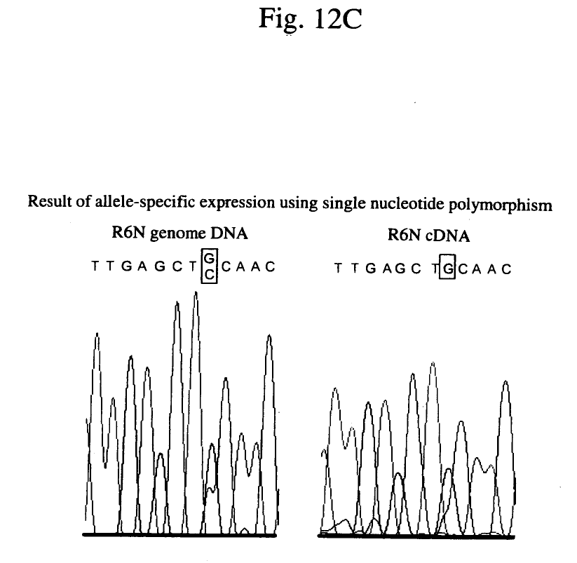

FIG. 12 shows allele-specific KIAA017 gene expression elucidating the function of the KIAA0172 gene. In the gene scan analysis, microsatellite analysis using a marker D9S1779 was performed, and it was shown that this marker site is deleted in cancer cells (FIG. 12A; allele loss result in the cancer tissue DNA in gene scan analysis). Furthermore, cDNAs from the normal tissue (R6N) and cancer tissue (R6T) were respectively analyzed by RT-PCR method. Although reference gene G6PDH was expressed in almost the same amount, KLAA017 gene was expressed only in the normal tissue (FIG. 12 B; gene expression loss result in the cancer tissue by RT-PCR method). Furthermore, it was revealed by comparing the genome polymorphisms (containing both the G sequence and C sequence ) with cDNA from the normal tissue that the expressed gene is only from one of the two alleles (one containing G sequence at the polymorphism site) in the normal tissue (FIG. 12C; allele-specific expression result using single nucleotide polymorphism). In the left panel of FIG. 12C, R6N genome DNA (TTGAGCT(G/C)CAAC) is examined while in the right panel, R6N cDNA (TTGAGCTGCAAC) is examined. Since the gene under such an allele—and cancer-specific suppressing control has not yet been found, it is considered that the information can be applied for cancer diagnosis.

(5) Analysis of Methylated Site

The degree of methylation in exon 1 region of the KIAA0 172 gene was determined by Sodium bisulphite method (Clark, S. J., Nucleic Acids Res. 22, 2990-2997 (1994)). Specifically, the genome DNA (125 ng) was first mixed with 2 μg of salmon sperm DNA, and denatured at 37° C. for 20 minutes in 0.3M NaOH. Cytosine residues were sulfurized by incubation in 5 M sodium bisulphite (Sigma) and 5 mM hydroquinone (Sigma) at 55° C. for 5 hours. The DNA sample was desalinated in a Qiagen column and desulfurized in 0.3M NaOH, and precipitated with ethanol. The thus processed DNA (30 ng) was amplified by PCR. PCR was conducted under a condition of 30 cycles of 94° C. for 30 seconds, 42° C. for 90 seconds and 72° C. for 1 minute. Two hundred fifty μM dNTP, Taq DNA polymerase and 914F primer AAGAAGAGA AAAGGTAGTTGG (SEQ ID No. 47) and 1413R primer CTATTAAAACTCAATTTCTTT (SEQ ID No. 48) were mixed and used in 50 μl (in total) of the reaction liquid. The PCR product was subjected to a semi-nested PCR using 914F primer and 1294R primer CCTAAAACCTCTATAATACACAAC (SEQ ID No. 49) under a condition of 25 cycles of 94° C. for 30 seconds, 52° C. for 1 minute and 72° C. for 1 minute. The PCR product was purified by QIA-quick PCR purification kit (Qiagen). The purified product was directly cloned into vector pGEM-T (Promega). The clone was selected at random and subjected to sequence analysis carried out by ABI 310 sequencer to confirm methylated sites.

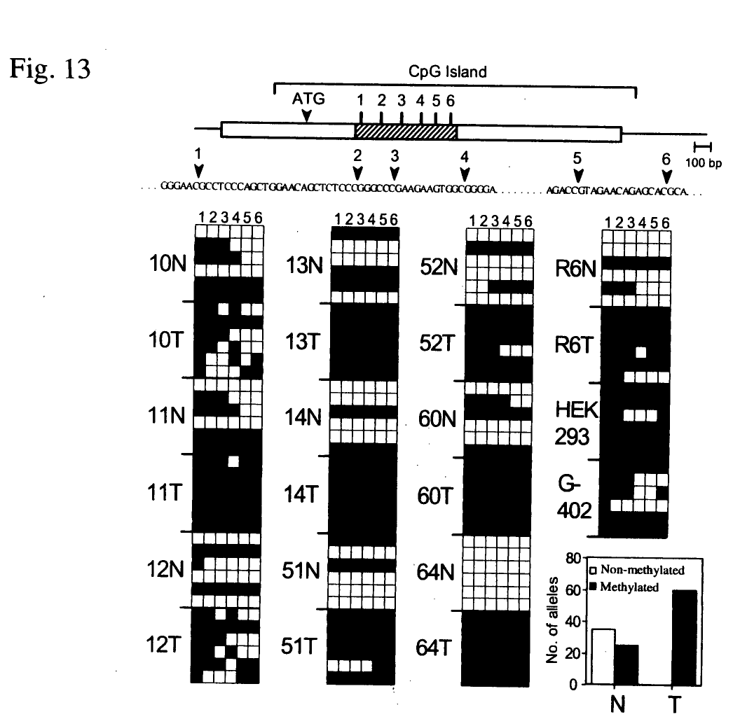

FIG. 13 shows a methylation pattern of the normal and cancer tissues and an established cancer cell line elucidating the function of the KIAA0172 gene. FIG. 13 is the result of investigating the methylation pattern using the sodium bisulfite method in CpG Island which exists in the 1st exon of the KIAA0172 gene where six CpG repeat therein. Methylated CpG sequences are indicated by solid box and non-methylated CpG sequences are indicated by open box for about 10 pairs of the normal (N) and cancer( T) tissue DNA obtained from patients (patient number is shown) and two types of established cell lines. The bar graph lower right in FIG. 13 shows the number of methylated and non-methylated alleles for the normal tissue and the established cell line. As is apparent from the drawing, it turns out that at least one site for each allele was methylated in the cancer tissue while in the normal tissue almost half for nine cases among ten cases have alleles which is not methylated at all with only one exception of 64N. Therefore, this gene has been already methylated in the normal tissue of renal cancer as well, and it is considered that diagnosis can be effected even in a normal state whether the risk is high or not. Furthermore, it became clear that all the alleles are already methylated for the established HEK293 cell and G-402 cell as well. When this result and the result of allele specificity are combined and considered, use of this gene can be useful for the inspection of risk factor.

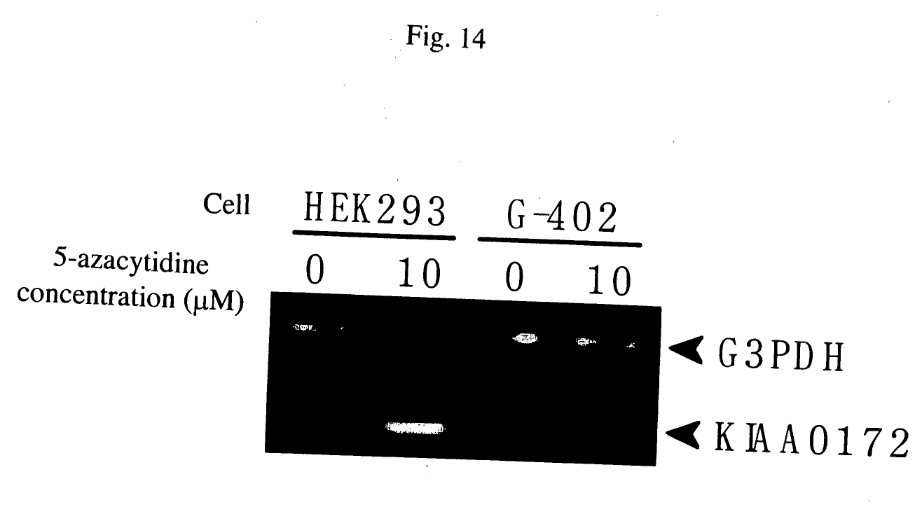

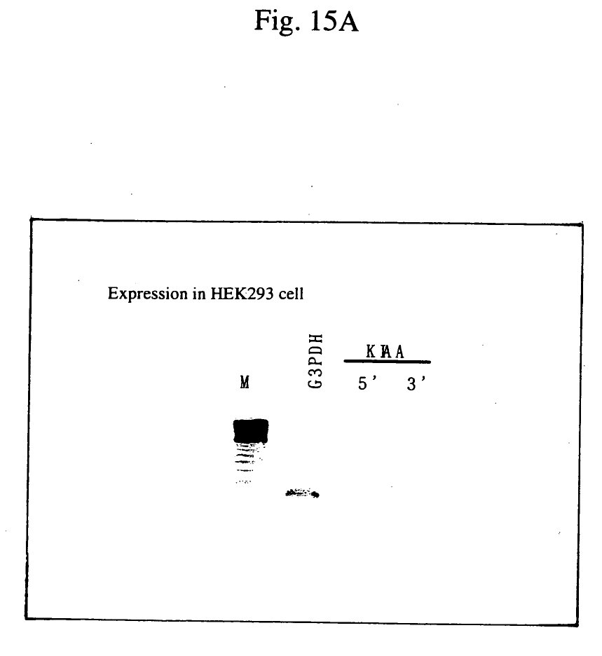

FIG. 14 shows activation of the gene expression by 5-aza-2′-deoxycytidine treatment elucidating the function of the KIAA0172 gene. As is shown in FIG. 14, when 5-aza-2′-deoxycytidine treatment was performed on two types of established cells in which expression of KIAAA0172 gene was not observed, expression was observed. Since this gene is not expressed in the established HEK293 and G-402 cells, it is supposed that demethylation takes place by 5-aza-2′-deoxycytidine treatment leading to re-expression. Therefore, the mechanism of inactivation of the gene was proved to be depended on methylation.

### EXAMPLE 4

### Functional Analysis of the KIAA0172 Gene

(1) Function for Suppressing Proliferation of Cancer Cell Line from Renal Cancer (part 1 :HEK293 Cells)

An experiment for suppressing proliferation was conducted by the following methods. The nucleotide sequence 340-4658 of the gene concerned was introduced into the vector pcDNA3.1 (pCMV-vec) (Invitrogen) to prepare a plasmid (pCMV-KIAA) for protein expression. This vector has a CMV promoter and constitutively expresses the gene inserted downstream. Moreover, since the vector contains a neomycin resistance gene, vectors can be selected by antibiotic neomycin. The gene product is a full length protein including codons for the translation initiator methionine and the termination of the translation.

5.1×10⁵ HEK293 cells were seeded on a 6 cm petri dish, and a transfection experiment was conducted. HEK293 cells were transfected with KIAA0172 expression vector (5 pg) using Lipofectamine 2000. Transfection with an empty vector (pcDNA3.1 (+)) was also effected as a control. After transfection, the cells was cultivated until they turned into 5x 103 cells. After two weeks of the transfection, they were treated with geneticin (500 μ/ml) and formation of colonies was confirmed. The obtained colonies were fixed and stained with Giemsa solution, and the number of the cells was measured. The stable HEK293 cell line colonies were isolated after selection with geneticin. It was confirmed by RT-PCR that these cells had expressed the KIAA0172 gene.

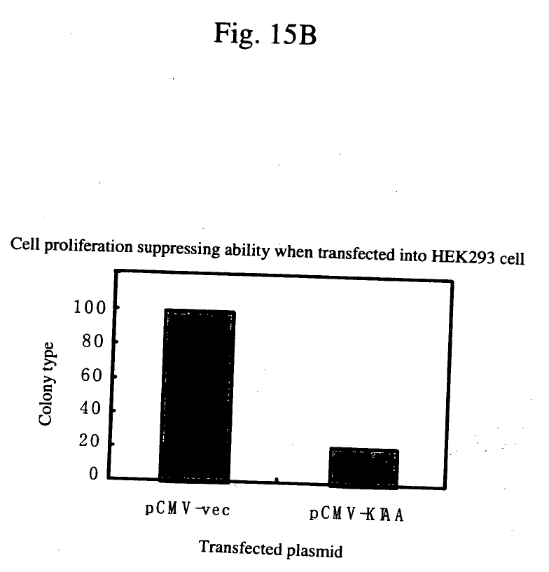

First, it was confirmed by RT-PCR that the gene concerned was not expressed in HEK293 cells. Next, the gene was inserted in the expression vector, introduced into a HEK293 cell which is an established renal cancer cell. When the gene concerned was made to express in this cell, suppression of proliferation of this cell was observed. That is, when the plasmid (pCMV-KIAA) which expresses the gene concerned was used, the colony formation rate was only 24% (FIG. 15; colony formation ability using HEK293 cell) as compared with the proliferation of the cell into which only the empty expression vector (pCMV-vec) was introduced as a control. FIG. 15 shows that expression of KIAA0172 gene is not observed in HEK293 cells (FIG. 15 A; expression in HEK293 cell) according to RT-PCR tests. When the number of colony is counted in the case where the expression plasmid (pCMV-KIAA) is introduced into the cell to constitutively express KIAA0172 and the case where only an empty vector is introduced as a control, the number decreased in the former case suggesting that cell proliferation is suppressed by the expression of this gene (FIG. 15 B; cell proliferation suppressing ability when transfected into HEK293 cell).

These results suggest that mutation in the gene concerned (also including a promoter region and other transcription regulation regions) suppress the expression and consequently induce carcinogenesis. Therefore, this is -a convincing proof which shows that the KIAA0172 is involved in the formation of renal cancer.

(2) The Suppressing Effect Against Proliferation of Cancer Cell Line Originated from Renal Cancer (part 2: G-402 cells)

The proliferation suppressing experiment was conducted by the above-mentioned method.

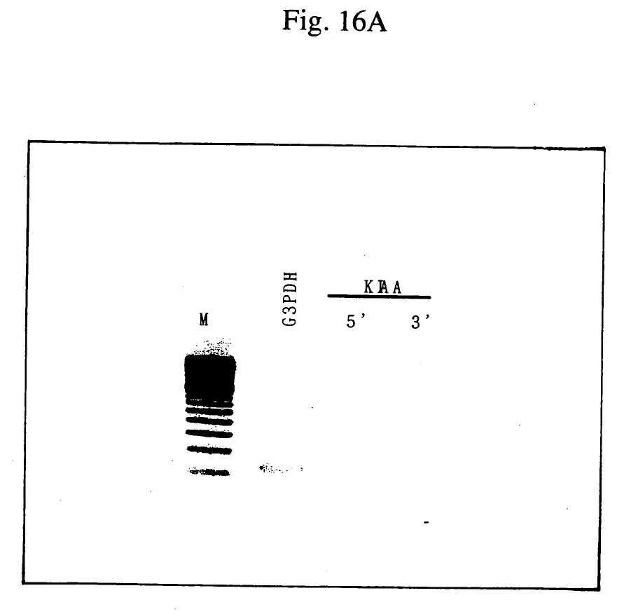

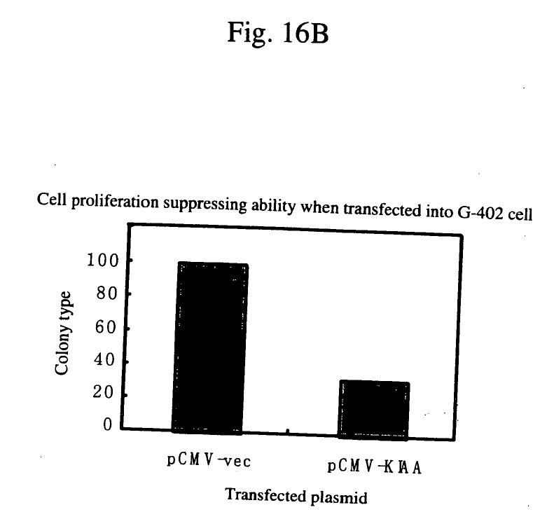

First, it was confirmed by RT-PCR that the gene concerned was not expressed in G-402 cells. Next, the gene was inserted into the expression vector, introduced into a G-402 cell which is an established renal cancer cell. When the gene concerned was made to express in this cell, suppression of proliferation of this cell was observed. That is, when the plasmid (pCMV-KIAA) which expresses the gene concerned was used, 30% of the colony formation rate was observed (FIG. 16) as compared with the proliferation of the cell into which only the expression vector (pCMV-vec) was introduced as a control. FIG. 16 shows by RT-PCR that expression of KIAA0172 gene is not observed in G-402 cells (FIG. 16 A). The case where the expression plasmid (pCMV-KIAA) is introduced into a cell, and this gene is constitutively expressed is compared with a case where only an empty vector is introduced as a control. Since the number of colonies decreases when the KIAA0172 gene is introduced, it is shown that cell proliferation is suppressed by the expression of this gene (FIG. 16 B; cell proliferation suppressing ability when transfected into G-402 cell).

It should be considered that the gene concerned (also including a promoter region and other transcription regulation regions) was mutated and as a result that the expression thereof was suppressed, carcinogenesis was induced in order to explain the above-mentioned phenomenon. Therefore, this is a convincing proof which shows that the gene concerned is involved in the formation of renal cancer.

### EXAMPLE 5

### Functional Analysis of the KIAA0172 Gene

Transformation Ability on the Cancer Cell Line from Renal Cancer (Cell Morphology Analysis)

The proliferation suppressing experiment was conducted by the above-mentioned method. The obtained cell was observed using an optical microscope.

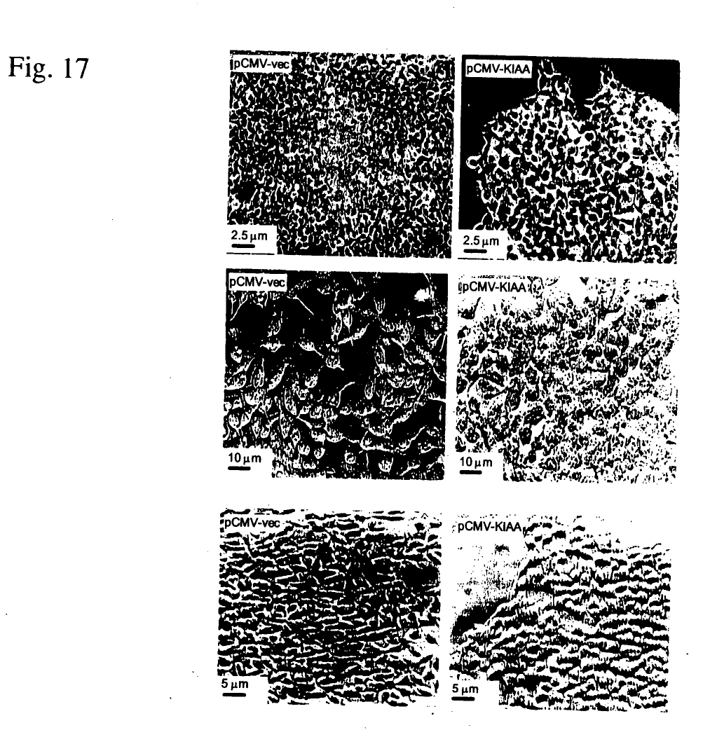

When the gene concerned was bound with the expression vector, introduced into HEK293 cell which is an established renal cancer cell and the gene concerned was made to express in this cell, change in the morphology of this cell was observed. That is, the cells in which the gene concerned was introduced and this gene was constitutively expressed exhibited increase in the adhesion area of the cells and the degree of adhesion between the cells were increased compared with a case where only an expression vector is introduced as a control (FIG. 17). The middle right in FIG. 17 is the cell into which was introduced the KIAA0172 gene. FIG. 17 compares the case where the KIAA0172 gene expression plasmid (pCMV-KIAA) is introduced into HEK293 cells and this gene is constitutively expressed with the case where only an empty vector is introduced as a control and shows that the cell morphology changes, when the KIAA0172 gene is introduced. The drawing showed the result which changed magnifying power etc., respectively.

These results suggest KIAA0172 has functions of controlling the proliferation of the gene and suppressing uncontrolled proliferation observed in cancer cells, and the proliferation suppression resulted in the change in cell morphology. The following two possibilities have not yet been excluded as causes of the cell growth suppression; at first interaction with growth factors may suppress cell growth, and second the direct effect on the cytoskeleton occurred during the immunostaining experiments using the anti-KIAA0172 antibody may also cause growth suppression.

### EXAMPLE 6

### Functional Analysis of the KIAA0172 Gene

(1) Functional Region Analysis (Part 1)

Homology search by the nucleotide sequence was performed by the following methods. Amino acid sequences which show homology to the amino acid sequence of the gene concerned (a total of 1194 residue) were searched using gene family search system GeneFIND available from Georgetown University, U.S. The software used was that described by Wu et al. (Bioinfomatics 14 volumes pages 223-224). As a result, human DAPK gene exhibited homology of 34.177% in the 157 amino-acid residues (amino-acid residues 1006-1162 of KIAA0172 and the amino-acid residues 481-630 of DAPK protein). This region included ankyrin repetition structure (FIG. 3).

As a result of gene homology search, it was found that the gene product concerned has significant homology to the functional site ankyrin repetition structure observed in many eukaryotic organism gene products. Although the ankyrin repetition structure was observed in many genes, the ankyrin homologous site for the gene showed particularly high homology to human DAPK gene product.

The above homology was about 34% on the amino acid sequence and is considered to be significant homology.

(2) Functional Region Analysis (Part 2)

Mutation (partial deletion) analysis was performed by the following methods. The gene containing a binding domain (or a part thereof) to a potentially functional site was cloned using MATCHMAKER GAL4 Two-Hybrid System 3 (a kit of Two-Hybrid method) of CLONTECH. A part of gene concerned containing the ankyrin homologous site (amino-acid residues 995-1194) was introduced into the DNA binding site derived from the GAL4 gene of the gene expression vector pGBKT7 and is introduced into yeast AH109 strain to express a fusion protein within the yeast. A plasmid (bait) containing both cDNA of the cDNA library of the kidney and AD site for GAL4 (amino-acid residues 768-881) was introduced into this yeast, and gene was made expressed. When the bait contains the gene product which interacts with ankyrin homologous site for the gene concerned, the AD site induces the GAL4 gene expression to exhibit a color. According to this method, cDNA derived from the gene (or a part thereof) which interacts with the ankyrin homologous site contained in the bait can be obtained as a clone.

Genes which exhibit interaction by Two-Hybrid method was searched using a variant which has only ankyrin homologous site for the gene concerned, and a cDNA from a plurality of genes (at least ten types) was obtained as a clone. Therefore, it became clear that it is the site to which this ankyrin homologous site interacts with the other gene products (proteins).

This method is one of the leading techniques to assay the interaction between proteins, and therefore it is believed that the information obtained was reliable.

### EXAMPLE 7

### Cell Proliferation Experiment Using Mouse

HEK293 cell line stably expressing the KIAA0172 gene were collected and suspended in a phosphate buffer solution. Then, a 3 to 4-week old BALB/c male nude-mouse was inoculated at one or two points of the flank with a suspension prepared so that it might become 5×10⁴ cells in 300 μl. Growth of the inoculated tumor cells was evaluated by measuring tumor capacity at a rate of twice per 10 days. All the animal experiments were conducted in accordance with the guideline of the laboratory.

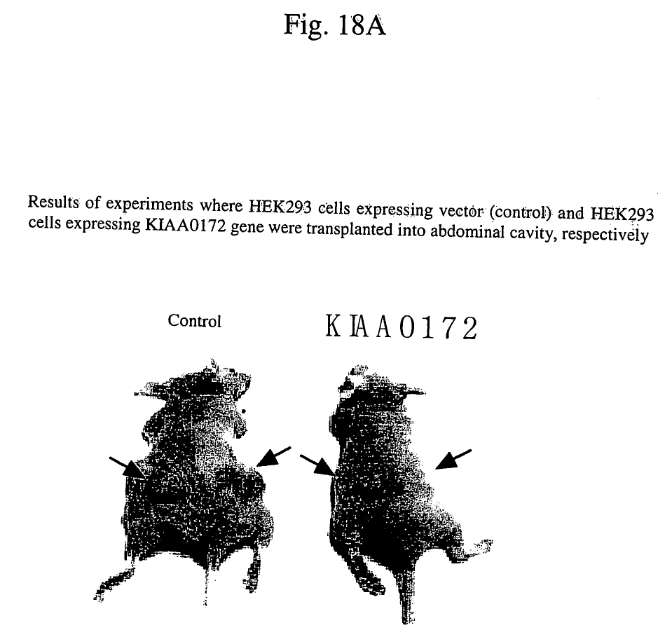

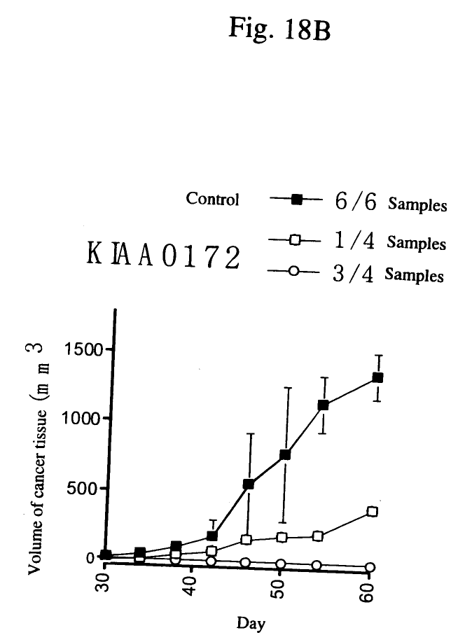

FIG. 18 shows the result of the cell proliferation suppression experiment using a nude mouse demonstrating the cell proliferation suppression ability of the KIAA0172 gene. FIG. 18 shows the result of observing the cell proliferation of HEK293 cells in which the KIAA0172 gene expression plasmid (pCMV-KIAA) was introduced and the gene was constitutively expressed. Only an empty vector was introduced in a control. As a result, it was made definite also in an experiment using a mouse that when the empty vector of control is used, cell proliferation is not suppressed to form cancer while in the case that the KIAA0 172 gene was expressed, cell proliferation is suppressed. The proliferation suppression effect of this gene on the cancer is made clear by this series of the cell proliferation suppression experiments, and application is envisaged not only in diagnosis but also in therapeutic treatment.

### INDUSTRIAL APPLICABILITY

The therapeutic agent of the present invention enables to treat cancer and the detecting agent of the present invention enables to detect cancer.

All the publications cited in the present specification are entirely incorporated in the present specification. It should be appreciated to those skilled in the art that various modifications and changes of the present invention can be effected within the technical concept of the appended claims and the scope of the present invention. The present invention intends to encompass such modifications and changes.

1. A method for detecting renal cancer by using a gene which encode the amino acid sequence represented by SEQ ID NO: 1 as a marker, which comprises a step of detecting the expression of a gene which encode the amino acid sequence represented by SEQ ID NO: 1 of a subject, wherein when little or no expression of the gene expression is detected it is judged that the possibility of the renal disease of the subject is high.

2. The method according to claim 1, wherein the expression of the gene which encode the amino acid sequence represented by SEQ ID NO: 1 is carried out by RT-PCR.

3. A method for detecting renal cancer by using a gene which encode the amino acid sequence represented by SEQ ID NO: 1 as a marker, which comprises a step of contacting a sample from a subject with a gene fragment of the gene which encode the amino acid sequence represented by SEQ ID NO: 1 having at least one of the following mutations of the gene (a) to (h):
(a) Mutation from CAC to CAG at the 52nd codon
(b) Mutation from GCG to GTG at the 168th codon
(c) Insertion of 6 nucleotides GCTGTA between the 268th and the 269th codons
(d) Mutation from GTA to GGA at the 269th codon
(e) Mutation from GAG to CAG at the 274th codon
(f) Mutation from TCC to GCC at the 306th codon
(g) Mutation from GCA to GTA at the 506th codon
(h) Mutation from CGT to CAT at the 509th codon; and
determining the presence of the mutation of the gene which encode the amino acid sequence represented by SEQ ID NO: 1 of the subject,
wherein the gene fragment has 15 to 100bp, and when the mutation of the gene of the subject is present it is judged that the possibility of the renal disease of the subject is high.

4. The method according to claim 1, wherein the determination of the presence of the mutation is carried out by PCR.
